(19) United States
(12) Patent Application Publication
(10) Pub. No.: US 2008/0146585 A1
(43) Pub. Date: Jun. 19, 2008

(54) Use Of C-Kit Inhibitors For Treating Inflammatory Muscle Disorders Including Myositis And Muscular Dystrophy

(75) Inventors: Alain Moussy, Paris (FR); Jean-Pierre Kinet, Lexington, MA (US)

Correspondence Address: FOLEY AND LARDNER LLP;SUITE 500, 3000 K STREET NW, WASHINGTON, DC, 20007, US

(73) Assignee: AB SCIENCE

(21) Appl. No.: 11/578,994

(22) PCT Filed: Apr. 19, 2005
(86) PCT No.: PCT/IB05/01367

§ 371 Date: Aug. 13, 2007

Related U.S. Application Data
(60) Provisional application No. 60/563,460, filed on Apr. 20, 2004.

(51) Int. Cl. A61K 31/497; A61K 31/4439; A61P 21/00

(52) U.S. Cl. 514/253.1; 514/342; 514/340

(57) ABSTRACT

The present invention relates to a method for treating inflammatory muscle disorders including myositis and muscular dystrophy comprising administering a compound capable of depleting mast cells or a compound inhibiting mast cell degranulation, to a human in need of such treatment. Such compounds can be chosen from c-kit inhibitors and more particularly non-toxic, selective and potent c-kit inhibitors. Preferably, said inhibitor is unable to promote death of IL-3 dependent cells cultured in presence of IL-3.

The present invention relates to a method for treating inflammatory muscle disorders including myositis and muscular dystrophy comprising administering a compound capable of depleting mast cells or a compound inhibiting mast cell degranulation, to a human in need of such treatment. Such compounds can be chosen from c-kit inhibitors and more particularly non-toxic, selective and potent c-kit inhibitors. Preferably, said inhibitor is unable to promote death of IL-3 dependent cells cultured in presence of IL-3.

The primary inflammatory muscle diseases comprise three main subsets: polymyositis (PM), dermatomyositis (DM) and inclusion body myositis (IBM). PM and DM are characterized by a proximal weakness that develops along weeks to months and by elevated creatine phosphokinase levels. Cutaneous involvement including both erythema and edema and infantile or adult onset are DM specific. PM and IBM only concern adults. Several PM/DM manifestations must be searched for because of their severity: swallowing disorders, various mechanisms of respiratory dysfunction (swallowing pneumopathies, interstitial lung disease, respiratory muscle deficiency) and cardiac involvement (Eymard, 2003).

The diagnosis for PM and DM consists mainly in two investigations, beside biopsy: muscle MRI imaging showing inflammatory pattern and specific detection of antisynthetase autoantibodies (PM/DM with interstitial lung disease) and anti-Mi-1 and 2 in DM (Eymard, 2003).

PM and DM differ in their histological and physiopathological characteristics: perivascular B and CD4 lymphocyte infiltrates and complement deposits at the origin of humoral induced vascular disease in DM and perimysial CD8 lymphocytes inducing a cellular mediated cytotoxic injury in PM. Class I HLA antigen expression on the muscle fibers and production of cytokines play a crucial role in the pathogenesis of these two diseases. PM and DM may be associated with cancers, connective-tissue disease (overlap syndrome). Some PMs are secondary to HIV, HTLV1 virus and toxoplasmosis infection (Eymard, 2003).

Inflammatory myopathies encompass a variety of syndromes with protean manifestations. Although the mainstay of therapy continues to include corticosteroids, there are a multitude of agents available for treating patients with myositis. These include many different immunosuppressive agents alone or in combination with each other, as well as an increasing array of novel and biologic agents targeting molecules implicated in the pathogenesis of inflammatory myopathy (Oddis, 2003).

The treatment principally relies on oral corticosteroid therapy, occasionally initiated via the intravenous route and which is active in 50 to 70% of cases. In patient with primary or secondary resistance, intolerance or dependence regarding corticosteroids, a second treatment line with immunosuppressive agents or intravenous immunoglobulin should be added (Cherin, 2003).

The systemic manifestations of myositis, particularly pulmonary involvement, are especially challenging to treat and contribute significantly to the morbidity and mortality of patients with polymyositis and dermatomyositis (Oddis, 2003).

Duchenne muscular dystrophy is the most common inherited lethal X-linked disorder of mankind and is caused by dystrophin deficiency. The steps involved in the dystrophin-deficiency-induced cellular and biochemical cascade which lead to myofibre necrosis, progressive muscle wasting in humans and dogs. In contrast prominent muscle hypertrophy occurs in mice and cats. The pathophysiology of this difference remains obscure. Dystrophin is an intracellular component of the membrane cytoskeleton and its absence would be expected to cause necrosis of isolated myofibres (cell autonomous defect). However, all dystrophin-deficient muscles characteristically show simultaneous degeneration of large groups of muscle fibers (grouped necrosis). This implies that cell death may be mediated by extracellular, non-cell autonomous factors which occur as a secondary consequence of dystrophin deficiency (Gorospe, 1994).

Dystrophin deficiency has been shown to be the underlying cause of Duchenne muscular dystrophy. Although dystrophin-deficient homologous animal models have been identified (dog, mouse, and cat), the clinical expression of the biochemical defect is species-specific. Thus, while the genetics and biochemistry of Duchenne dystrophy is understood, the pathophysiological cascade leading to muscle weakness remains obscure (Gorospe, 1994).

The development of therapeutic strategies that overcome the unique problems raised by Duchenne muscular dystrophy (DMD) has led to the development of many contemporary approaches to human disease in general. Various treatment approaches have been explored such as pharmacological therapies and cell-based, cytokine, and genetic therapies that are all targeted to specific features of dystrophic DMD muscle pathology. In genetic therapies, the large size of the dystrophin gene has necessitated the development and use of novel functional minidystrophin and microdystrophin genes, muscle-specific promoter systems, and gutted adenoviral systems.

Nevertheless, as of today, the pathogenesis of PM and DM is not yet totally resolved (Dalakas, 2003) and there are no cure available. In DM it is hypothesized that a putative antibody directed against endothelial cells activate complement C3. Activated C3 leads to formation of C3b and lytic component of the complement pathway. The complement deposits induce swollen endothelial cells vacuolisation, capillary necrosis perivascular inflammation, ischemia and destruction of muscle fibres. In PM, CD8 positive cells invade MHC-1 antigen expressing muscle fibres. CD 8 cells may release their granule contents including perforine and granzyme to induce apoptosis and necrosis of muscle fibers. In this process it is interesting to note that muscle fibres abnormally express MHC class I and II antigens, and costimulatory molecules.

In both PM and DM the role of innate immune cells has not be investigated, and particularly the role of mast cells.

Mast cells (MC) are tissue elements derived from a particular subset of hematopoietic stem cells that express CD34, c-kit and CD13 antigens (Kirshenbaum, 1999 and Ishizaka, 1993). Mast cells produce a large variety of mediators categorized into three groups: preformed granule-associated mediators (histamine, proteoglycans, and neutral proteases), lipid-derived mediators (prostaglandins, thromboxanes and leucotrienes), and various cytokines (IL-1, UL-2, IL-3, IL-4, IL-5, UL-6, UL-8, TNF-α, GM-CSF, MIP-1α, MIP-1β and IFN-γ) most of them having strong pro-inflammatory activities. For instance, a massive release of MCs mediators is responsible for anaphylactic reactions that could be sometimes fatal to the patients and are always responsible for a significant morbidity. Since MCs are distributed in almost all the body sites, hypersecretion of mediators by activated elements can lead to multiple organ failures.

In this context, we have tested a tyrosine kinase inhibitor in murine models of these diseases, and have unexpectedly shown a dramatic effect to prevent diseases and reduce the severity of the symptoms. We believe that mast cells could be attracted by activated complement proteins and then may produce chemokines, metalloprotease and cytokines to recruit other cells of the immune system and allow their passage through the vascular wall. Cytokines and chemokines are also released and may allow immune cells to pass through endothelial cells. CD4 T cells and B cells are the main cells observed in the inflammatory infiltrate. Furthermore mast cells may participate to produce toxic mediators resulting in muscle fibers apoptosis and may also induce fibrosis through the production of TGF-β and PDGF-R.

In connection with the present invention, we have discovered that c-kit inhibitors could reduce inflammatory muscle disorders and degeneration of the skeletal and voluntary muscles. Such inhibitors also lower the production of chemokines, metalloprotease and cytokines secreted by mast cells to recruit other cells of the immune system and allow their passage through the vascular wall as well as toxic mediators resulting in muscle fibres apoptosis.

### DESCRIPTION

The present invention relates to a method for treating inflammatory muscle disorders including myositis and muscular dystrophy, comprising administering a compound capable of depleting mast cells or a compound inhibiting mast cells degranulation in a human in need of such treatment.

Said method for treating inflammatory muscle disorders including myositis and muscular dystrophy can comprise administering a c-kit inhibitor to a human in need of such treatment.

Preferred compounds are c-kit inhibitor, more particularly a non-toxic, selective and potent c-kit inhibitor.

Such inhibitors can be selected from the group consisting of 2-(3-Substitutedaryl)amino-4-aryl-thiazoles such as 2-(3-amino)arylamino-4-aryl-thiazoles, 2-aminoaryloxazoles, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating inflammatory muscle disorders including myositis and muscular dystrophy comprising administering a non toxic, potent and selective c-kit inhibitor is a pyrimidine derivatives, more particularly N-phenyl-2-pyrimidine-amine derivatives of formula I:

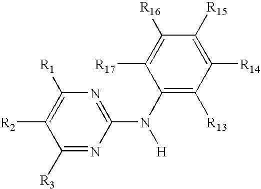

wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II:

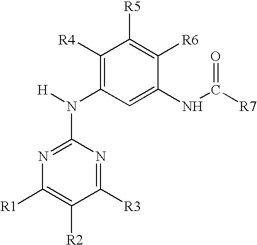

Wherein R1, R2 and R3 are independently chosen from H, F, Cl, Br, L a C1-C5 alkyl or a cyclic or heterocyclic group, especially a pyridyl group;

R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

Preferably, R7 is the following group:

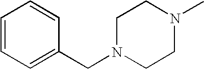

Among these compounds, the preferred are defined as follows:

R1 is a heterocyclic group, especially a pyridyl group,

### R2 and R3 are H,

R4 is a C1-C3 alkyl, especially a methyl group,

### R5 and R6 are H,

and R7 is a phenyl group bearing at least one substituent, which in turn possesses at least one
basic site, such as an amino function, for example the group:

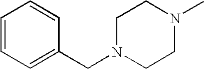

Therefore, in a preferred embodiment, the invention relates to a method for treating inflammatory muscle disorders including myositis and muscular dystrophy comprising the administration of an effective amount of the compound known in the art as CGP57148B:

4-(4-méthylpipérazine-1-ylméthyl)-N-[4-méthyl-3-(4-pyridine-3-yl)pyrimidine-2ylamino)phényl]-benzamide corresponding to the following formula:

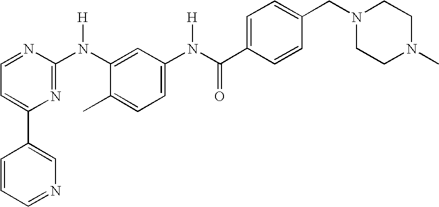

The preparation of this compound is described in example 21 of EP 564 409 and the β-form, which is particularly useful is described in WO 99/03854.

In another preferred embodiment, the invention contemplates the method mentioned above, wherein said c-kit inhibitor is selected from 2-(3-Substitutedaryl)amino-4-aryl-thiazoles such as those for which the applicant filed PCT/IB2005/000401, incorporated herein by reference, especially compounds of formula III:

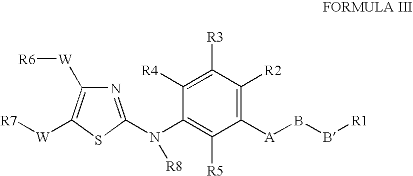

wherein
R⁶ and R⁷ are independently from each other chosen from one of the following:
i) hydrogen, a halogen (selected from F; Cl, Br or I),
ii) an alkyl¹ group defined as a linear, branched or cycloalkyl group containing from 1 to 10 carbon atoms, or from 2 or 3 to 10 carbon atoms, (for example methyl, ethyl, propyl, butyl, pentyl, hexyl . . . ) and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl;
(iii) an aryl¹ group defined as phenyl or a substituted variant thereof bearing any combination, at any one ring position, of one or more substituents such as
halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   (iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   (v) trifluoromethyl, carboxyl, cyano, nitro, formyl, hydroxy, N(alkyl¹)(alkyl¹), and amino, the latter nitrogen substituents optionally in the form of a basic nitrogen functionality.
   R⁸ is one of the following:
   (i) hydrogen, or
   (ii) a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
   (iii) CO—R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, as well as trifluoromethyl, C₁₋₆alkyloxy, carboxyl, cyano, nitro, formyl, hydroxy, C₆₋₄alkylamino, di(C₁₋₆alkyl)amino, and amino, the latter nitrogen substituents optionally in the form of a basic nitrogen functionality, as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R2, R3, R4 and R5 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, as well as trifluoromethyl, C₁₋₆alkyloxy, amino, C₁₋₆alkylamino, di(C₁₋₆alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

### A is: CH2, O, S, SO2, CO, or COO,

B is a bond or NH, NCH3, NR*, (CH2)n (n is 0, 1 or 2), O, S, SO2, CO, or COO,
B′ is a bond or NH, NCH3, NR*, (CH2)n (n is 0, 1 or 2), O, S, SO2, CO or COO;
R* being an alkyl¹, aryl¹ or heteroaryl¹
W is a bond or a linker selected from NH, NHCO, NHCOO, NHCONH, NHSO2, NHSO2NH, CO, CONH, COO, COCH2, (CH2)n (n is 0, 1 or 2), CH2-CO, CH2COO, CH2-NH, O, OCH2, S, SO2, and SO2NH

### R¹ is:

a) a linear or branched alkyl group containing from 1 to 10 carbon atoms optionally substituted with at least one heteroatom, notably a halogen selected from I, Cl, Br and F, and/or bearing a pendant basic nitrogen functionality;
b) an aryl or heteroaryl group optionally substituted by an alkyl or aryl group optionally substituted with a heteroatom, notably a halogen selected from I, Cl, Br and F or bearing a pendant basic nitrogen functionality
c) an alkyl¹, aryl¹ or heteroaryl¹.

It will be understood that a C1-C10 alkyl encompasses a methyl, ethyl, propyl, and a C2 to C4 alkyl or a C2 to C10 alkyl.

For example, a subset of compounds may correspond to

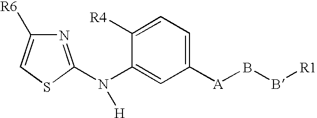

Wherein R1, R4 and R6 have the meaning as defined above.

It will be understood that A-B—B′ includes but is not limited to:

### CH2, CH2-CO, CH2-CO—CH2, CH2COO, CH2-CH2-CO, CH2-CH2-COO, CH2-NH, CH2-CH2-NH, CH2-NH—CH2 or CH2-NH—CO or CH2-CO—NH

It will be understood that A-B—B′ also includes but is not limited to:

### CO—CH2, COO—CH2, CO—CH2-CH2, CO—NH, or CO—NH—CH2

as well as O—CH2

It will also be understood that NH in B or B′ can also be NCH3

In the above formula III, when W is other than a single bond, it will be understood that A can be also be NH or NCH3.

In the above formula, the following combinations are contemplated:
R6 is (iv), R4 is H or CH3, A-B—B′ is CO—NH and R1 is as defined above.
R6 is (iv), R4 is H or CH₃, A-B—B′ is CH2-CO—NH and R1 is as defined above.
R6 is (iv), R4 is H or CH3, A-B—B′ is CH2-CO and R1 is as defined above.
R6 is (iv), R4 is H or CH3, A-B—B′ is CH2-NH—CO and R1 is as defined above.
R6 is (iv), R4 is H or CH3, A-B—B′ is CH2-NH and R1 is as defined above.
R6 is (iv), R4 is H or CH3, A-B—B′ is CH2 and R1 is as defined above.
R6 is W-(iv), R4 is a C₁-C₂ alkyl, A-B—B′ is CO—NH and R1 is as defined above.
R6 is (iv), R4 is a C₁-C₂ alkyl, A-B—B′ is CH2-CO—NH and R1 is as defined above.
R6 is (iv), R4 is a C₁-C₂ alkyl, A-B—B′ is CH2-CO and R1 is as defined above.
R6 is a pyridyl according to (iv), R4 is a C₁-C₂ alkyl, A-B—B′ is CO—NH, CH2-CO—NH, CH2-CO, CH2-NH, CH2-NH—CO and R1 is as defined above.

In the above combination, R1 can be an alkyl¹.

In the above combination, R1 can be an aryl¹.

In the above combination, R1 can be an heteroaryl¹.

In another preferred embodiment, the invention contemplated the method mentioned above, wherein said c-kit inhibitor is selected from 2-(3-amino)arylamino-4-aryl-thiazoles such as those for which the applicant filed WO 2004/014903, incorporated herein in the description, especially compounds of formula IV:

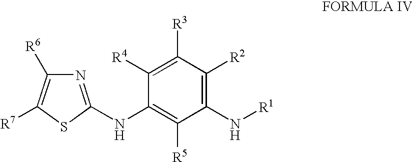

and wherein R¹ is:
a) a linear or branched alkyl group containing from 1 to 10 carbon atoms optionally substituted with at least one heteroatom, notably a halogen selected from I, Cl, Br and F, and/or bearing a pendant basic nitrogen functionality;
b) an aryl or heteroaryl group optionally substituted by an alkyl or aryl group optionally substituted with a heteroatom, notably a halogen selected from I, Cl, Br and F or bearing a pendant basic nitrogen functionality;
c) a —CO—NH—R, —CO—R, —CO—OR or a —CO—NRR′ group, wherein R and R′ are independently chosen from H or an aryl, heteroaryl, alkyl and cycloalkyl group optionally substituted with at least one heteroatom, notably a halogen selected from I, Cl, Br and F, and/or bearing a pendant basic nitrogen functionality;
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy,
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality;
and R⁷ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another preferred embodiment, when R¹ has the meaning depicted in c) above, the invention is directed to compounds of the following formulas:

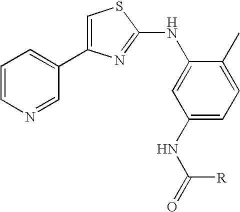

wherein R is H or an organic group that can be selected for example from a linear or branched alkyl group containing from 1 to 10 carbon atoms optionally substituted with at least one heteroatom or bearing a pendant basic nitrogen functionality, a cycloalkyl, an aryl or heteroaryl group optionally substituted by an alkyl, a cycloalkyl, an aryl or heteroaryl group optionally substituted with a heteroatom, notably a halogen selected from I, Cl, Br and F and/or bearing a pendant basic nitrogen functionality.

Among the particular compounds in which R1 has the meaning as depicted in c) above, the invention is directed to amide-aniline, amide-benzylamine, amide-phenol, urea compounds of the following formulas respectively:

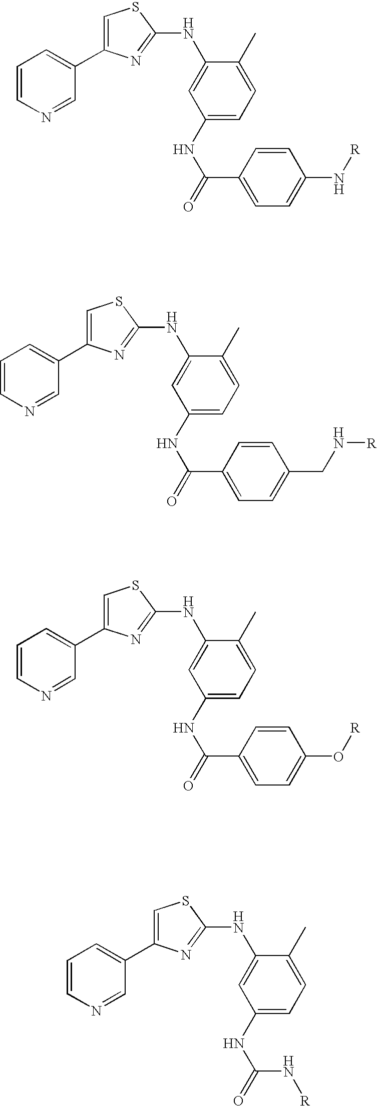

wherein R is H or an organic group that can be selected for example from a linear or branched alkyl group containing from 1 to 10 carbon atoms optionally substituted with at least one heteroatom or bearing a pendant basic nitrogen functionality; a cycloalkyl, an aryl or heteroaryl group optionally substituted with a heteroatom, notably a halogen selected from L Cl, Br and F and/or bearing a pendant basic nitrogen functionality; or a a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;
a —SO2-R group wherein R is an alkyl, cycloalkyl, aryl or heteroaryl optionally substituted with an heteroatom, notably a halogen selected from I, Cl, Br and F and/or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.

Among the particular compounds in which R1 has the meaning as depicted in a) and b) above, the invention is directed to N-Aminoalkyl-N-thiazol-2-yl-benzene-1,3-diamine compounds of the following formula IVbis:

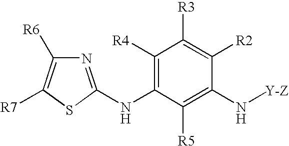

wherein Y is a linear or branched alkyl group containing from 1 to 10 carbon atoms;
wherein Z represents an aryl or heteroaryl group, optionally substituted at one or more ring position with any permutation of the following groups:
a halogen such as F, Cl, Br, I;
a linear or branched alkyl group containing from 1 to 10 carbon atoms optionally substituted with at least one heteroatom (for example a halogen) and/or bearing a pendant basic nitrogen functionality, a cycloalkyl, an aryl or heteroaryl group optionally substituted with at least one heteroatom, notably a halogen selected from I, Cl, Br and F, and/or bearing a pendant basic nitrogen functionality, or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an O—R, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality, or a cycloalkyl, an aryl or heteroaryl group substituted by an alkyl, a cycloalkyl, an aryl or heteroaryl group optionally substituted with an heteroatom, notably a halogen selected from L Cl, Br and F, and/or bearing a pendant basic nitrogen functionality;
an NRaRb, where Ra and Rb represents a hydrogen, or a linear or branched alkyl group containing from 1 to 10 carbon atoms optionally substituted with at least one heteroatom (for example a halogen) and I or bearing a pendant basic nitrogen functionality or a cycle; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a COOR, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
a CONRaRb, where Ra and Rb are a hydrogen or a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCOR, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCOOR, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NHCONRaRb, where Ra and Rb are a hydrogen or a linear or branched alkyl group containing from 1 to 10 carbon atoms optionally substituted with at least one heteroatom (for example a halogen) and/or bearing a pendant basic nitrogen functionality, a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an OSO₂R, where R is a linear or branched alkyl group containing from 1 to 10 carbon atoms optionally substituted with at least one heteroatom (for example a halogen) and/or bearing a pendant basic nitrogen functionality; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
an NRaOSO₂Rb, where Ra and Rb are a linear or branched alkyl group containing from 1 to 10 carbon atoms optionally substituted with at least one heteroatom (for example a halogen) and/or bearing a pendant basic nitrogen functionality; Ra can also be a hydrogen; a cycloalkyl, an aryl or heteroaryl group optionally substituted with at least one heteroatom, notably a halogen selected from I, Cl, Br and F, and/or bearing a pendant basic nitrogen functionality; or a cycloalkyl, an aryl or heteroaryl group substituted by an alkyl, a cycloalkyl, an aryl or heteroaryl group optionally substituted with an heteroatom, notably a halogen selected from I, Cl, Br and F, and/or bearing a pendant basic nitrogen functionality;
   R² is hydrogen, halogen or a linear or branched alkyl group containing from 1 to 10 carbon atoms, trifluoromethyl or alkoxy;
   R³ is hydrogen, halogen or a linear or branched alkyl group containing from 1 to 10 carbon atoms, trifluoromethyl or alkoxy;
   R⁴ is hydrogen, halogen or a linear or branched alkyl group containing from 1 to 10 carbon atoms, trifluoromethyl or alkoxy;
   R⁵ is hydrogen, halogen or a linear or branched alkyl group containing from 1 to 10 carbon atoms, trifluoromethyl or alkoxy;
   R⁶ is one of the following:
   (i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
   (ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
   (iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
   iv) H, a halogen selected from I, F, Cl or Br, NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality, and R⁷ is one of the following:
   (i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
   (ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
   (iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
   iv) H, an halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

It will be understood that a C1-C10 alkyl encompasses a methyl, ethyl, propyl, and a C2 to C4 alkyl or a C2 to C₁₀ alkyl.

An example of preferred compounds of the above formula is depicted below:

4-{[Methyl-3-(4-pyridin-3-yl-thiazol-2-ylamino)-phenylamino]-methyl}-benzoic acid methyl ester

Among the compounds of formula III or IV, the invention is particularly embodied by the compounds of the following formula V:

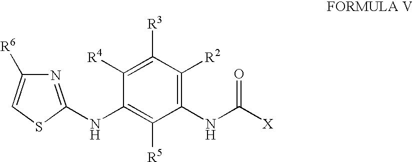

wherein X is R or NRR′ and wherein R and R′ are independently chosen from H, an aryl, a heteroaryl, an alkyl, or a cycloalkyl group optionally substituted with at least one heteroatom, such as for example a halogen chosen from F, I, Cl and Br and optionally bearing a pendant basic nitrogen functionality; or an aryl, a heteroaryl, an alkyl or a cycloalkyl group substituted with an aryl, a heteroaryl, an alkyl or a cycloalkyl group optionally substituted with at least one heteroatom, such as for example a halogen chosen from F, I, Cl and Br and optionally bearing a pendant basic nitrogen functionality,
R² is hydrogen, halogen or a linear or branched alkyl group containing from 1 to 10 carbon atoms, trifluoromethyl or alkoxy;
R³ is hydrogen, halogen or a linear or branched alkyl group containing from 1 to 10 carbon atoms, trifluoromethyl or alkoxy;
R⁴ is hydrogen, halogen or a linear or branched alkyl group containing from 1 to 10 carbon atoms, trifluoromethyl or alkoxy;
R⁵ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br, NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R⁶, which in the formula n is connected to position 4 of the thiazole ring, may instead occupy position 5 of the thiazole ring.

Among the preferred compounds corresponding formula III, IV or V, the invention is directed to compounds in which R1 or X is a substituted alkyl, aryl or heteroaryl group bearing a pendant basic nitrogen functionality represented for example by the structures a to f and g to m shown below, wherein the wavy line corresponds to the point of attachment to core structure of formula III, IV or V:

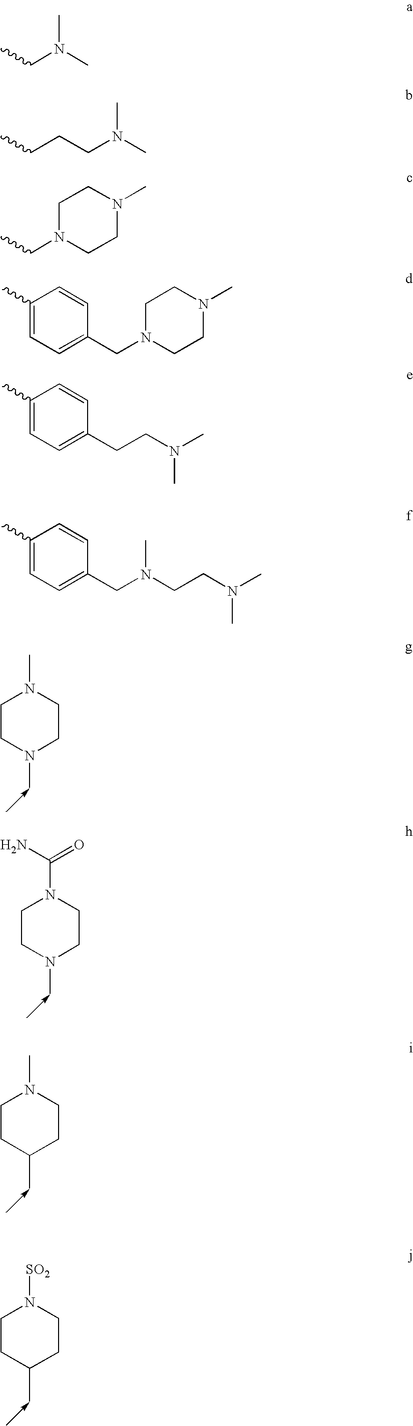

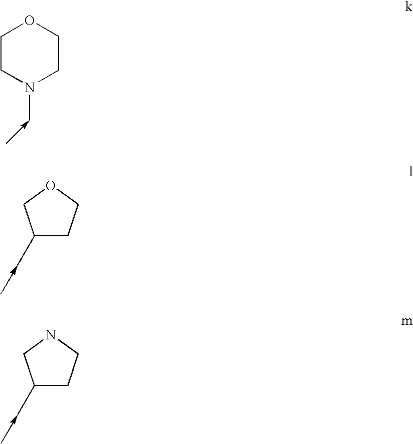

Among group a to f, is preferentially group d. Also, for g to m, the arrow may include a point of attachment to the core structure via a phenyl group.

Furthermore, among the preferred compounds of formula III, IV or V, the invention concerns the compounds in which R² and R³ are hydrogen. Preferentially, R⁴ is a methyl group and R⁵ is H. In addition, R⁶ is preferentially a 3-pyridyl group (cf. structure g below), or a 4-pyridyl group (cf. structure h below) or a benzonitrile group. The wavy line in structure g and h correspond to the point of attachment to the core structure of formula III, IV or V.

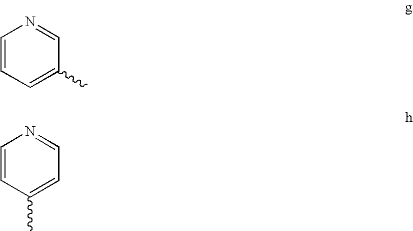

Alternatively, among the preferred compounds of formula III, IV or V, the invention concerns the compounds in which R⁶ or R⁷ is preferentially a cyanophenyl group as shown below, wherein the wavy line in structure p and q correspond to the point of attachment to the core structure of formula A, IV or V:

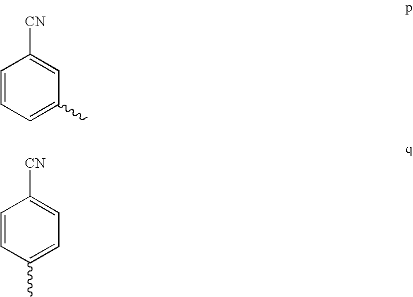

In one particular embodiment, R1 in formula III and IV, X in formula V and Z in formula IVbis can be:

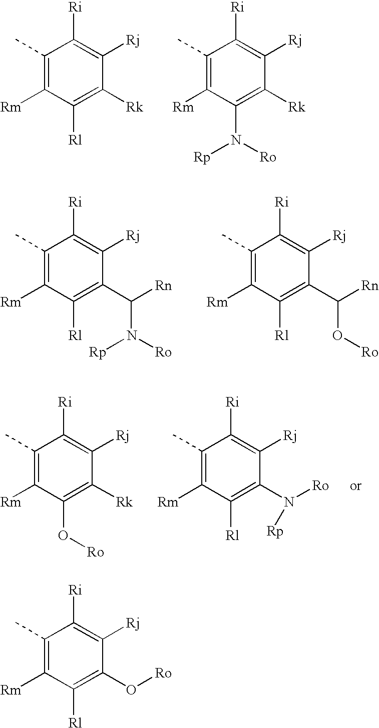

wherein Ri, Rj, Rk, Rl, Rm, Ro, and Rp are independently chosen from:
H, an halogen such as Cl, F, Br, I; a trifluoromethyl group, a CN group, SO2, OH, or a group selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality, or a cycloalkyl, an aryl or heteroaryl group optionally substituted with a cycloalkyl, an aryl or heteroaryl group optionally substituted with an heteroatom, notably a halogen selected from I, CL Br and F or bearing a pendant basic nitrogen functionality;
a NRR′, NRCOR, NRCONR′R″, NROSO₂R′, SO2-R, COOR, CONRR′, NHCOOR, CO—R, CO—NRR′, OR or OSO₂R group where R and R′ are independently chosen from H or a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality, or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality.

For example, one of Ri, Rj, Rk, Rl, Rm, Ro or Rp is selected from group a, b, c, g, h, i, j, k, l, m as defined above such as Rk is one of a, b, c, g, h, i, j, k, l, m and Ri, Rj, Rl, Rm is H.

Thus, the invention contemplates:
1—A compound of formula V as depicted above, wherein X is group d and R⁶ is a 3-pyridyl group.
2-A compound of formula V as depicted above, wherein X is group d and R⁴ is a methyl group.
3—A compound of formula III or IV as depicted above, wherein R¹ is group d and R² and/or R³ and/or R⁵ is H.
4—A compound of formula III or IV as depicted above, wherein R⁶ is a 3-pyridyl group and R⁴ is a methyl group.
5—A compound of formula III or IV as depicted above, wherein R² and/or R³ and/or R⁵ is H and R⁴ is a methyl group.
6—A compound of formula III or IV as depicted above wherein R² and/or R³ and/or R⁵ is H, R⁴ is a methyl group and R⁶ is a 3-pyridyl group.

Among the compounds of formula IV, the invention is particularly embodied by the compounds wherein R², R³, R⁵ are hydrogen, corresponding to the following formula

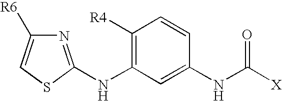

wherein X is R or NRR′ and wherein R and R′ are independently chosen from H or an organic group that can be selected for example from a linear or branched alkyl group containing from 1 to 10 carbon atoms optionally substituted with at least one heteroatom or bearing a pendant basic nitrogen functionality, a cycloalkyl, an aryl or heteroaryl group optionally substituted with an heteroatom, notably a halogen selected from I, Cl, Br and F or bearing a pendant basic nitrogen functionality; or a cycloalkyl, an aryl or heteroaryl group optionally substituted with a cycloalkyl, an aryl or heteroaryl group optionally substituted with an heteroatom, notably a halogen selected from I, Cl, Br and F or bearing a pendant basic nitrogen functionality;
a —SO2-R group wherein R is an alkyl, cycloalkyl, aryl or heteroaryl optionally substituted with a heteroatom, notably a halogen selected from I, Cl, Br and F or bearing a pendant basic nitrogen functionality, or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, and/or bearing a pendant basic nitrogen functionality.
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

In another alternative, substituent R6, which in the formula III is connected to position 4 of the thiazole ring, may instead occupy position 5 of the thiazole ring.

### EXAMPLES

2-(2-methyl-5-amino)phenyl-4-(3-pyridyl)-thiazole
4-(4-Methyl-piperazin-1-ylmethyl)-N-[3-(4-pyridin-3-yl-thiazol-2-ylamino)-phenyl]-benzamide
N-[4-Methyl-3-(4-phenyl-thiazol-2-ylamino)-phenyl]-4-(4-methyl-piperazin-1-ylmethyl)-benzamide
N-[3-([2,4′]Bithiazolyl-2′-ylamino)-4-methyl-phenyl]-4-(4-methyl-piperazin-1-ylmethyl)-benzamide
4-(4-Methyl-piperazin-1-ylmethyl)-N-[4-methyl-3-(4-pyrazin-2-yl-thiazol-2-ylamino)-phenyl]-benzamide
2-[5-(3-Iodo-benzoylamino)-2-methyl-phenylamino]-thiazole-4-carboxylic acid ethyl ester
2-{2-Methyl-5-[4-(4-methyl-piperazin-1-ylmethyl)-benzoylamino]-phenylamino}-thiazole-4-carboxylic acid ethyl ester
2-(2-chloro-5-amino)phenyl-4-(3-pyridyl)-thiazole
3-Bromo-N-{3-[4-(4-chloro-phenyl)-5-methyl-thiazol-2-ylamino]-4-methyl-phenyl}-benzamide
{3-[4-(4-Chloro-phenyl)-5-methyl-thiazol-2-ylamino]-4-methyl-phenyl}-carbamic acid isobutyl ester
2-[5-(3-Bromo-benzoylamino)-2-methyl-phenylamino]-5-(4-chloro-phenyl)-thiazole-4-carboxylic acid ethyl ester
2-[5-(3-Bromo-benzoylamino)-2-methyl-phenylamino]-5-(4-chloro-phenyl)-thiazole-4-carboxylic acid (2-dimethylamino-ethyl)-amide
N-{3-[4-(4-Methoxy-phenyl)-thiazol-2-ylamino]-4-methyl-phenyl}-4-(4-methyl-piperazin-1ylmethyl)-benzamide
4-(4-Methyl-piperazin-1-ylmethyl)-N-{4-methyl-3-[4-(3-trifluoromethyl-phenyl)-thiazol-2-ylamino]-phenyl}-benzamide
N-{4-Methyl-3-[4-(3-nitro-phenyl)-thiazol-2-ylamino]-phenyl}-4-(4-methyl-piperazin-1-ylmethyl)-benzamide
N-{3-[4-(2,5-Dimethyl-phenyl)-thiazol-2-ylamino]-4-methyl-phenyl}-4-(4-methyl-piperazin-1-ylmethyl)-benzamide
N-{3-[4-(4-Chloro-phenyl)-thiazol-2-ylamino]-4-methyl-phenyl}-4-(4-methyl-piperazin-1-ylmethyl)-benzamide
N-{3-[4-(3-Methoxy-phenyl)-thiazol-2-ylamino]-4-methyl-phenyl}-4-(4-methyl-piperazin-1-ylmethyl)-benzamide
N-[4-Methyl-3-(4-pyridin-3-yl-thiazol-2-ylamino)-phenyl]-isonicotinamide
2,6-Dichloro-N-[4-methyl-3-(4-pyridin-3-yl-thiazol-2-ylamino)-phenyl]-isonicotinamide
3-Phenyl-propynoic acid [4-methyl-3-(4-pyridin-3-yl-thiazol-2-ylamino)-phenyl]-amide
Cyclohexanecarboxylic acid [4-methyl-3-(4-pyridin-3-yl-thiazol-2-ylmethyl)-phenyl]-amide
5-[4-Methyl-3-(4-pyridin-3-yl-thiazol-2-ylamino)-phenylcarbamoyl]-pentanoic acid ethyl ester
1-Methyl-cyclohexanecarboxylic acid [4-methyl-3-(4-pyridin-3-yl-thiazol-2-ylmethyl)-phenyl]-amide
4-tert-Butyl-cyclohexanecarboxylic acid [4-methyl-3-(4-pyridin-3-yl-thiazol-2-ylamino)-phenyl]-amide
N-[4-Methyl-3-(4-pyridin-3-yl-thiazol-2-ylamino)-phenyl]-4-morpholin-4-yl-butyramide

Among the compounds of formula IV, the invention is particularly embodied by the compounds wherein X is a urea group, a —CO—NRR′ group, corresponding to the [3-(thiazol-2-ylamino)-phenyl]-urea family and the following formula:

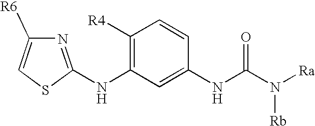

wherein Ra, Rb are independently chosen from Y-Z as defined above or
H or an organic group that can be selected for example from a linear or branched alkyl group containing from 1 to 10 carbon atoms optionally substituted with at least one heteroatom and/or bearing a pendant basic nitrogen functionality; a cycloalkyl, an aryl or heteroaryl group optionally substituted with a heteroatom, notably a halogen selected from I, Cl, Br and F or bearing a pendant basic nitrogen functionality, or a cycloalkyl, an aryl or heteroaryl group optionally substituted with a cycloalkyl, an aryl or heteroaryl group optionally substituted with a heteroatom, notably a halogen selected from I, Cl, Br and F or bearing a pendant basic nitrogen functionality;
a —SO2-R group wherein R is an alkyl, cycloalkyl, aryl or heteroaryl optionally substituted with an heteroatom, notably a halogen selected from I, Cl, Br and F or bearing a pendant basic nitrogen functionality; or a —CO—R or a —CO—NRR′ group, wherein R and R′ are independently chosen from H, an alkyl, a cycloalkyl, an aryl or heteroaryl group optionally substituted with at least one heteroatom, notably selected from I, Cl, Br and F, or bearing a pendant basic nitrogen functionality.
R⁴ is hydrogen, halogen or a linear or branched alkyl group containing from 1 to 10 carbon atoms, trifluoromethyl or alkoxy;
R⁶ is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, and alkoxy;
(ii) a heteroaryl group such as a 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy.
iv) H, a halogen selected from I, F, Cl or Br; NH2, NO2 or SO2-R, wherein R is a linear or branched alkyl group containing one or more group such as 1 to 10 carbon atoms, and optionally substituted with at least one heteroatom, notably a halogen selected from I, Cl, Br and F, and/or bearing a pendant basic nitrogen functionality.

### Example 1

1-(4-Methoxy-phenyl)-3-[4-methyl-3-(4-pyridin-3-yl-thiazol-2-ylamino)-phenyl]-urea
1-(4-Bromo-phenyl)-3-[4-methyl-3-(4-pyridin-3-yl-thiazol-2-ylamino)-phenyl]-urea
1-[4-Methyl-3-(4-pyridin-3-yl-thiazol-2-ylamino)-phenyl]-3-(4-trifluoromethyl-phenyl)-urea
1-(4-Fluoro-phenyl)-3-[4-methyl-3-(4-pyridin-3-yl-thiazol-2-ylamino)-phenyl]-urea
1-[4-Methyl-3-(4-pyridin-3-yl-thiazol-2-ylamino)-phenyl]-3-(3,4,5-trimethoxy-phenyl)-urea
4-{3-[4-Methyl-3-(4-pyridin-3-yl-thiazol-2-ylamino)-phenyl]-ureido}-benzoic acid ethyl ester
1-[4-Methyl-3-(4-pyridin-3-yl-thiazol-2-ylamino)-phenyl]-3-thiophen-2-yl-urea
1-Cyclohexyl-1-(N-Cyclohexyl-formamide)-3-[4-methyl-3-(4-pyridin-3-yl-thiazol-2-ylamino)-phenyl]-urea
1-(2,4-Dimethoxy-phenyl)-3-[4-methyl-3-(4-pyridin-3-yl-thiazol-2-ylamino)-phenyl]-urea
1-(2-Iodo-phenyl)-1-(N-(2-Iodo-phenyl)-formamide)-3-[4-methyl-3-(4-pyridin-3-yl-thiazol-2-ylamino)-phenyl]-urea
1-(3,5-Dimethyl-isoxazol-4-yl)-3-[4-methyl-3-(4-pyridin-3-yl-thiazol-2-ylamino)-phenyl]-urea
1-(2-Iodo-phenyl)-3-[4-methyl-3-(4-pyridin-3-yl-thiazol-2-ylamino)-phenyl]-urea
1-(4-Difluoromethoxy-phenyl)-3-[4-methyl-3-(4-pyridin-3-yl-thiazol-2-ylamino)-phenyl]-urea
1-(4-Dimethylamino-phenyl)-3-[4-methyl-3-(4-pyridin-3-yl-thiazol-2-ylamino)-phenyl]-urea
1-(2-Fluoro-phenyl)-3-[4-methyl-3-(4-pyridin-3-yl-thiazol-2-ylamino)-phenyl]-urea
1-(2-Chloro-phenyl)-3-[4-methyl-3-(4-pyridin-3-yl-thiazol-2-ylamino)-phenyl]-urea
1-(3-Fluoro-phenyl)-3-[4-methyl-3-(4-pyridin-3-yl-thiazol-2-ylamino)-phenyl]-urea
1-[4-Methyl-3-(4-pyridin-3-yl-thiazol-2-ylamino)-phenyl]-3-p-tolyl-urea
3-Bromo-N-[4-methyl-3-(4-pyridin-3-yl-thiazol-2-ylamino)-phenyl]-benzamide
3-Iodo-N-[4-methyl-3-(4-pyridin-3-yl-thiazol-2-ylamino)-phenyl]-benzamide
4-Hydroxymethyl-N-[4-methyl-3-(4-pyridin-3-yl-thiazol-2-ylamino)-phenyl]-benzamide
4-Amino-N-[4-methyl-3-(4-pyridin-3-yl-thiazol-2-ylamino)-phenyl]-benzamide
2-Iodo-N-[4-methyl-3-(4-pyridin-3-yl-thiazol-2-ylamino)-phenyl]-benzamide
4-Iodo-N-[4-methyl-3-(4-pyridin-3-yl-thiazol-2-ylamino)-phenyl]-benzamide
4-(3-{4-[4-Methyl-3-(4-pyridin-3-yl-thiazol-2-ylamino)-phenylcarbamoyl]-phenyl}-ureido)-benzoic acid ethyl ester
N-[4-Methyl-3-(4-pyridin-3-yl-thiazol-2-ylamino)-phenyl]-4-[3-(4-trifluoromethyl-phenyl)-ureido]-benzamide
4-[3-(4-Bromo-phenyl)-ureido]-N-[4-methyl-3-(4-pyridin-3-yl-thiazol-2-ylamino)-phenyl]-benzamide
4-Hydroxy-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-(3-thiophen-2-yl-ureido)-benzamide
4-[3-(3,5-Dimethyl-isoxazol-4-yl)-ureido]-N-[4-methyl-3-(4-pyridin-3-yl-thiazol-2-ylamino)-phenyl]-benzamide
4-[3-(4-Methoxy-phenyl)-ureido]-N-[4-methyl-3-(4-pyridin-3-yl-thiazol-2-ylamino)-phenyl]-benzamide
4-[3-(4-Difluoromethoxy-phenyl)-ureido]-N-[4-methyl-3-(4-pyridin-3-yl-thiazol-2-ylamino)-phenyl]-benzamide
Thiophene-2-sulfonic acid 4-[4-methyl-3-(4-pyridin-3-yl-thiazol-2-ylamino)-phenylcarbamoyl]-phenyl ester
4-Iodo-benzenesulfonic acid 4-[4-methyl-3-(4-pyridin-3-yl-thiazol-2-ylamino)-phenylcarbamoyl]-phenyl ester
N-[4-Methyl-3-(4-pyridin-3-yl-thiazol-2-ylamino)-phenyl]-4-(thiophene-2-sulfonylamino)-benzamide
3-Fluoro-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-pyridin-4-yl-benzamide
4-Dimethylamino-N-[4-methyl-3-(4-pyridin-3-yl-thiazol-2-ylamino)-phenyl]-benzamide
2-Fluoro-5-methyl-N-[4-methyl-3-(4-pyridin-3-yl-thiazol-2-yl no)-phenyl]-benzamide
4-tert-Butyl-N-[4-methyl-3-(4-pyridin-3-yl-thiazol-2-ylamino)-phenyl]-benzamide
4-Isopropoxy-N-[4-methyl-3-(4-pyridin-3-yl-thiazol-2-ylmethyl)-phenyl]-benzamide
Benzo[1,3]dioxole-5-carboxylic acid [4-methyl-3-(4-pyridin-3-yl-thiazol-2-ylmethyl)-phenyl]-amide
N-[4-Methyl-3-(4-pyridin-3-yl-thiazol-2-ylamino)-phenyl]-3-(2-morpholin-4-yl-ethoxy)-benzamide
N-[4-Methyl-3-(4-pyridin-3-yl-thiazol-2-ylmethyl)-phenyl]-4-pyridin-4-yl-benzamide
3-Cyano-N-[4-methyl-3-(4-pyridin-3-yl-thiazol-2-ylamino)-phenyl]-benzamide
2-Fluoro-N-[4-methyl-3-(4-pyridin-3-yl-thiazol-2-ylamino)-phenyl]-3-trifluoromethyl-benzamide
3-Fluoro-benzenesulfonic acid 4-[4-methyl-3-(4-pyridin-3-yl-thiazol-2-ylamino)-phenylcarbamoyl]-phenyl ester
4-Aminomethyl-N-[4-methyl-3-(4-pyridin-3-yl-thiazol-2-ylamino)-phenyl]-benzamide
2-Fluoro-benzenesulfonic acid 4-[4-methyl-3-(4-pyridin-3-yl-thiazol-2-ylamino)-phenylcarbamoyl]-phenyl ester
3-Methoxy-N-[4-methyl-3-(4-pyridin-3-yl-thiazol-2-ylmethyl)-phenyl]-benzamide
4-(4-Methyl-piperazin-1-yl)-N-[4-methyl-3-(4-pyridin-3-yl-thiazol-2-ylmethyl)-phenyl]-benzamide
3-Methyl-N-[4-methyl-3-(4-pyridin-3-yl-thiazol-2-ylamino)-phenyl]-benzamide
Biphenyl-3-carboxylic acid [4-methyl-3-(4-pyridin-3-yl-thiazol-2-ylamino)-phenyl]-amide
N-[4-Methyl-3-(4-Pyridin-3-yl-thiazol-2-ylamino)-phenyl]-3-trifluoromethyl-benzamide
N-[4-Methyl-3-(4-pyridin-3-yl-thiazol-2-ylamino)-phenyl]-4-pyrrolidin-1-ylmethyl-benzamide
4-[3-(2,4-Dimethoxy-phenyl)-ureido]-N-[4-methyl-3-(4-pyridin-3-yl-thiazol-2-ylamino)-phenyl]-benzamide
4-[3-(2-Iodo-phenyl)-ureido]-N-[4-methyl-3-(4-pyridin-3-yl-thiazol-2-ylamino)-phenyl]-benzamide
4-[3-(4-Fluoro-phenyl)-ureido]-N-[4-methyl-3-(4-pyridin-3-yl-thiazol-2-ylamino)-phenyl]-benzamide
3-Bromo-4-methyl-N-[4-methyl-3-(4-pyridin-3-yl-thiazol-2-ylamino)-phenyl]-benzamide
4-Fluoro-N-[4-methyl-3-(4-pyridin-3-yl-thiazol-2-ylamino)-phenyl]-benzamide 4-Cyano-N-[4-methyl-3-(4-pyridin-3-yl-thiazol-2-ylamino)-phenyl]-benzamide
4-Fluoro-N-[4-methyl-3-(4-pyridin-3-yl-thiazol-2-ylamino)-phenyl]-benzamide

### Example 2

4-(4-methyl-piperazin-1-ylmethyl)-N-[4-methyl-3-(4-pyridin-3-yl-thiazol-2-ylamino)-phenyl]-benzamide
3,5-Dibromo-4-(4-methyl-piperazin-1-ylmethyl)-N-[4-methyl-3-(4-pyridin-3-yl-thiazol-2-ylamino)-phenyl]-benzamide
4-Diethylaminomethyl-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-morpholin-4-ylmethyl-benzamide
4-Dipropylaminomethyl-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-4-piperidin-1-ylmethyl-benzamide
4-[(Diisopropylamino)-methyl]-N-[4-methyl-3-(4-pyridin-3-yl-thiazol-2-ylamino)-phenyl]-benzamide
{4-[4-Methyl-3-(4-pyridin-3-yl-thiazol-2-ylamino)-phenylcarbamoyl]-benzyl}-carbamic acid tert-butyl ester
3-Fluoro-4-(4-methyl-piperazin-1-ylmethyl)-N-[4-methyl-3-(4-pyridin-3-yl-thiazol-2-ylamino)-phenyl]-benzamide
4-(4-Methyl-piperazin-1-ylmethyl)-N-[4-methyl-3-(4-pyridin-3-yl-thiazol-2-ylmethyl)-phenyl]-3-trifluoromethyl-benzamide
2,3,5,6-Tetrafluoro-4-(4-methyl-piperazin-1-ylmethyl)-N-[4-methyl-3-(4-pyridin-3-yl-thiazol-2-ylamino)-phenyl]-benzamide
N-{3-[4-(4-Fluoro-phenyl)-thiazol-2-ylamino]-4-methyl-phenyl}-4-(4-methyl-piperazin-1-ylmethyl)-benzamide
3-Bromo-4-(4-methyl-piperazin-1-ylmethyl)-N-[4-methyl-3-(4-pyridin-3-yl-thiazol-2-ylamino)-phenyl]-benzamide
3-Chloro-4-(4-methyl-piperazin-1-ylmethyl)-N-[4-methyl-3-(4-pyridin-3-yl-thiazol-2-ylamino)-phenyl]-benzamide
4-(4-Methyl-piperazin-1-ylmethyl)-N-[4-methyl-3-(4-pyridin-4-yl-thiazol-2-ylamino)-phenyl]-benzamide
N-{3-[4-(4-Cyano-phenyl)-thiazol-2-ylamino]-4-methyl-phenyl}-4-(4-methyl-piperazin-1-ylmethyl)-benzamide
4-[1-(4-Methyl-piperazin-1-yl)-ethyl]-N-[4-methyl-3-(4-pyridin-3-yl-thiazol-2-ylmethyl)-phenyl]-benzamide
4-(1-Methoxy-ethyl)-N-[4-methyl-3-(4-pyridin-3-yl-thiazol-2-ylmethyl)-phenyl]-benzamide
N-{4-Methyl-3-[4-(5-methyl-pyridin-3-yl)-thiazol-2-ylamino]-phenyl}-4-(4-methyl-piperazin-1-ylmethyl)-benzamide
3-Iodo-4-(4-methyl-piperazin-1-ylmethyl)-N-[4-methyl-3-(4-pyridin-3-yl-thiazol-2-ylmethyl)-phenyl]-benzamide
N-[4-Methyl-3-(4-pyridin-3-yl-thiazol-2-ylamino)-phenyl]-4-[3-(4-trifluoromethyl-phenyl)-ureidomethyl]-benzamide
3,5-Dibromo-N-[4-methyl-3-(4-pyridin-3-yl-thiazol-2-ylamino)-phenyl]-4-[(3-morpholin-4-yl-propylamino)-methyl]-benzamide
3,5-Dibromo-N-[4-methyl-3-(4-pyridin-3-yl-thiazol-2-amino)-phenyl]-4-piperidin-1-ylmethyl-benzamide
4-(4-Methyl-piperazin-1-ylmethyl)-N-[4-methyl-3-(4-pyridin-2-yl-thiazol-2-ylamino)-phenyl]-benzamide
N-{3-[4-(3-Fluoro-phenyl)-thiazol-2-ylamino]-4-methyl-phenyl}-4-(4-methyl-piperazin-1-ylmethyl)-benzamide
N-{3-[4-(2-Fluoro-phenyl)-thiazol-2-ylamino]-4-methyl-phenyl}-4-(4-methyl-piperazin-1-ylmethyl)-benzamides

### Example 3

3-Dimethylamino-N-[4-methyl-3-(4-pyridin-3-yl-thiazol-2-ylamino)-phenyl]-benzamide
3-(4-Methyl-piperazin-1-yl)-N-[4-methyl-3-(4-pyridin-3-yl-thiazol-2-ylamino)-phenyl]-benzamide
N-[4-Methyl-3-(4-pyridin-3-yl-thiazol-2-ylamino)-phenyl]-3-morpholin-4-yl-benzamide
Among the compounds of formula IV, the invention is particularly embodied by the compounds wherein X is a —OR group, corresponding to the family [3-(Thiazol-2-ylamino)-phenyl]-carbamate and the following formula IV-6

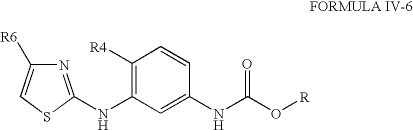

wherein R is independently chosen from an organic group that can be selected for example from a linear or branched alkyl group containing from 1 to 10 carbon atoms optionally substituted with at least one heteroatom and/or bearing a pendant basic nitrogen functionality, a cycloalkyl, an aryl or heteroaryl group optionally substituted with an heteroatom, notably a halogen selected from I, Cl, Br and F and/or bearing a pendant basic nitrogen functionality; or a cycloalkyl, an aryl or heteroaryl group optionally substituted with a cycloalkyl, an aryl or heteroaryl group optionally substituted with a heteroatom, notably a halogen selected from I, Cl, Br and F and/or bearing a pendant basic nitrogen functionality;
R4 and R6 are as defined above.

In still another preferred embodiment, the invention contemplated the method mentioned above, wherein said c-kit inhibitor is selected from 2-aminoaryloxazoles of formula X:

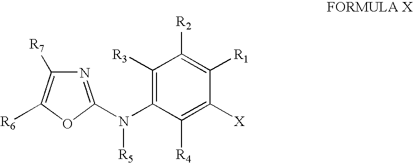

wherein substituents R1-R7 and X are defined as follows:
R1, R2, R3 and R4 each independently are selected from hydrogen, halogen (selected from F, Cl, Br or I), a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, amino, C₁₋₆alkylamino, di(C₁₋₄alkyl)amino, carboxyl, cyano, nitro, formyl, hydroxy, and CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.

R5 is one of the following:

(i) hydrogen, or
(ii) a linear or branched alkyl group containing from i to 10 carbon atoms and optionally substituted with one or more hetereoatoms such as halogen (selected from F, Cl, Br or I), oxygen, and nitrogen, the latter optionally in the form of a pendant basic nitrogen functionality, or
(iii) CO—R8 or COOR8 or CONHR8 or SO2R8 wherein R8 may be
a linear or branched alkyl group containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, or
an aryl group such as phenyl or a substituted variant thereof bearing any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality, as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a pendant basic nitrogen functionality, as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality, or
a heteroaryl group such as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as halogen (selected from F, Cl, Br or I), alkyl groups containing from 1 to 10 carbon atoms and optionally substituted with one or more hetereoatoms such as halogen (selected from F, Cl, Br or I), oxygen, and nitrogen, the latter optionally in the form of a pendant basic nitrogen functionality; as well as trifluoromethyl, C₁₋₆alkyloxy, carboxyl, cyano, nitro, formyl, hydroxy, C₁₋₆alkylamino, di(C₁₋₆alkyl)amino, and amino, the latter nitrogen substituents optionally in the form of a basic nitrogen functionality; as well as CO—R, COO—R, CONH—R, SO2-R, and SO2NH—R wherein R is a linear or branched alkyl group containing from 1 to 10 carbon atoms and optionally substituted with at least one heteroatom, notably a halogen (selected from F, Cl, Br or I), oxygen, and nitrogen, the latter optionally in the form of a pendant basic nitrogen functionality.
   R6 and R7 each independently are selected from:
   i) hydrogen, a halogen (selected from F, Cl, Br or I), or
   ii) an alkyl¹ group defined as a linear, branched or cycloalkyl group containing from 1 to 10 carbon atoms and optionally substituted with one or more hetereoatoms such as halogen (selected from F, Cl, Br or I), oxygen, and nitrogen (the latter optionally in the form of a pendant basic nitrogen functionality); as well as trifluoromethyl, carboxyl, cyano, nitro, formyl; as well as CO—R, COO—R, CONH—R, SO2-R, and SO2NH—R wherein R is a linear or branched alkyl group containing 1 to 10 carbon atoms and optionally substituted with at least one heteroatom, notably a halogen (selected from F, Cl, Br or I), oxygen, and nitrogen, the latter optionally in the form of a pendant basic nitrogen functionality; as well as a cycloalkyl or aryl or heteroaryl group optionally substituted by a pendant basic nitrogen functionality, or
   (iii) an aryl¹ group defined as phenyl or a substituted variant thereof bearing any combination, at any one ring position, of one or more substituents such as
halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, or
   (iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which may additionally bear any combination, at any one ring position, of one or more substituents such as
halogen (selected from F, Cl, Br or I);
an alkyl¹ group;
a cycloalkyl, aryl or heteroaryl group optionally substituted by a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, or
   (v) an O-aryl¹, or NH-aryl¹, or O-heteroaryl¹ or NH-heteroaryl¹ group
   (vi) trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality, or
   (vi) NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl.

### X is:

NR9R10, wherein R9 and or R10 are hydrogen or:
   i) an alkyl¹ group, CF3 or
   ii) an aryl¹, heteroaryl¹ or cycloalkyl group optionally substituted by a pendant basic nitrogen functionality, or
   iii) a CO—R, COO—R, CON—RR′ or SO2-R, where R and R′ are a hydrogen, alkyl, aryl or heteroaryl¹, optionally substituted by a a pendant basic nitrogen functionality; or:
CO—NR9R10, wherein R9 and/or R10 are hydrogen or:
   i) an alkyl¹ group, CF3 or
   ii) an aryl¹, heteroaryl¹ or cycloalkyl group optionally substituted by a a pendant basic nitrogen functionality.

Such compound may be selected from N-Aminoalkyl-N′-oxazol-2-yl-benzene-1,3-diamines of the following formula:

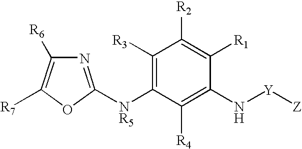

wherein R5=H, Y is a linear or branched alkyl group containing from 1 to 10 carbon atoms and Z represents an aryl or a heteroaryl group, optionally substituted by a pendant basic nitrogen functionality.

For example, it is the 4-{[4-Methyl-3-(4-pyridin-3-yl-oxazol-2-ylamino)-phenylamino]-methyl}-benzoic acid methyl ester.

The above 2-aminoaryloxazoles compounds may have the formula XI:

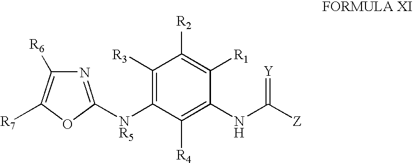

Wherein R5 is H, Y is selected from O, S and Z corresponds to H, alkyl, or NRR′, wherein R and R′ are independently chosen from H or alkyl or aryl¹ or heteroaryl¹, optionally substituted by a pendant basic nitrogen functionality, for example:

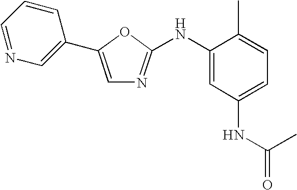

or a compound of formula XI-1:

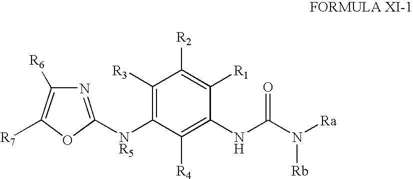

wherein Ra, Rb are independently chosen from H or alkyl¹ or aryl¹ or heteroaryl¹, optionally substituted by a pendant basic nitrogen functionality, for example:

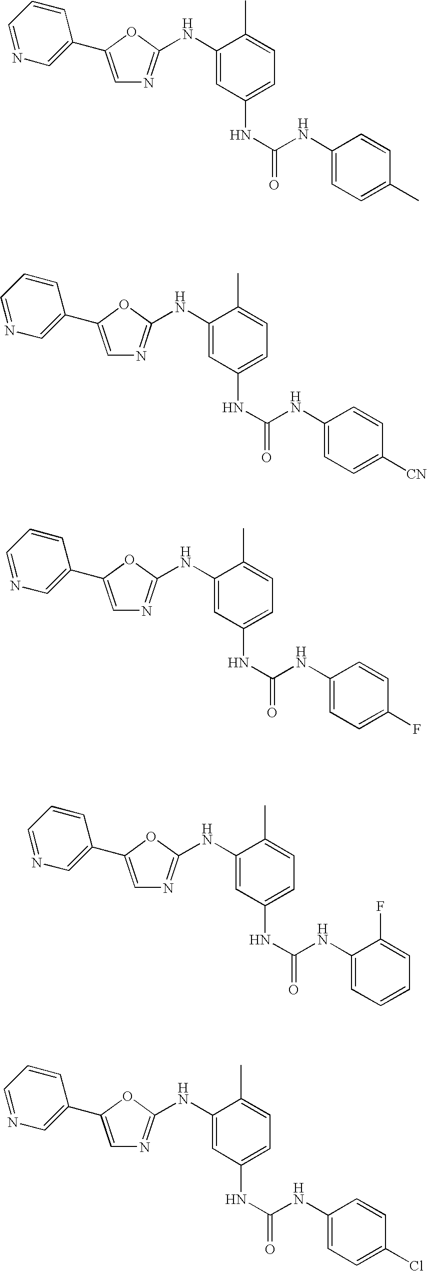

or a compound of formula XI-2:

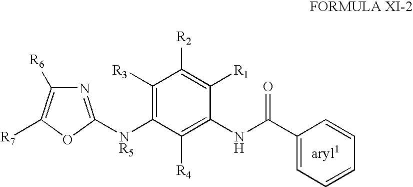

wherein R5=H, Z is an aryl¹ group, aryl¹ being selected from:
a phenyl or a substituted variant thereof bearing any combination, at any one ring position, of one or more substituents such as
halogen (selected from I, F, Cl or Br);
an alkyl¹ group;
a cycloalkyl, aryl or heteroaryl group optionally substituted by a pendant basic nitrogen functionality;
trifluoromethyl, O-alkyl¹, carboxyl, cyano, nitro, formyl, hydroxy, NH-alkyl¹, N(alkyl¹)(alkyl¹), and amino, the latter nitrogen substituents optionally in the form of a basic nitrogen functionality;
   NHCO—R or NHCOO—R or NHCONH—R or NHSO2-R or NHSO2NH—R or CO—R or COO—R or CONH—R or SO2-R or SO2NH—R wherein R corresponds to hydrogen, alkyl¹, aryl or heteroaryl, for example

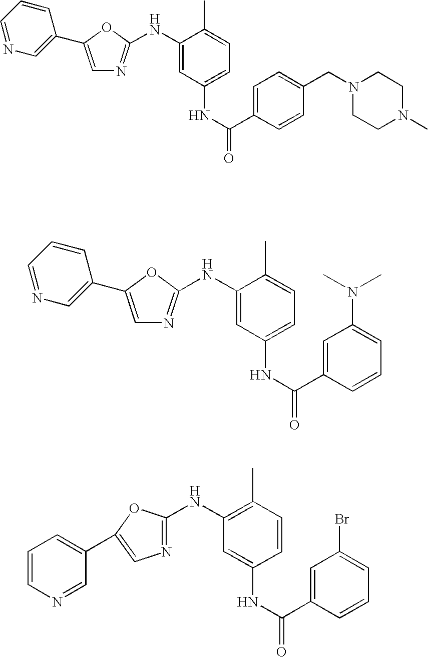

or a compound of formula XI-3:

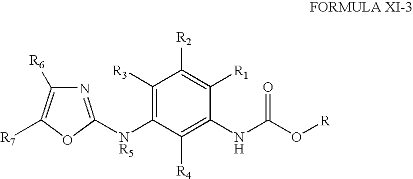

wherein R5=H and R is independently alkyl¹, aryl¹ or heteroaryl¹ as defined above.

### Examples of Compounds of Formula X:

4-{[4-Methyl-3-(4-pyridin-3-yl-oxazol-2-ylamino)-phenylamino]-methyl}-benzoic acid methyl ester
4-Methyl-N1-(5-pyridin-3-yl-oxazol-2-yl)-N3-(5-pyridin-4-yl-oxazol-2-yl)-benzene-1,3-diamine m.p.
4-Methyl-N1-(5-phenyl-oxazol-2-yl)-N3-(5-pyridin-4-yl-oxazol-2-yl)-benzene-1,3-diamine
4-Methyl-N1-(5-phenyl-[1,3,4]oxadiazol-2-yl)-N3-(5-pyridin-4-yl-oxazol-2-yl)-benzene-1,3-diamine
N1-Benzooxazol-2-ylmethyl-N3-(5-pyridin-4-yl-oxazol-2-yl)-benzene-1,3-diamine
N-[4-Methyl-3-(5-pyridin-4-yl-oxazol-2-ylamino)-phenyl]-C-phenyl-methanesulfon-amide
N-[4-Methyl-3-(5-pyridin-3-yl-oxazol-2-ylamino)-phenyl]-acetamide
2-Cyano-N-[4-methyl-3-(5-pyridin-4-yl-oxazol-2-ylamino)-phenyl]-acetamide
2-Ethoxy-N-[4-methyl-3-(5-pyridin-3-yl-oxazol-2-ylamino)-phenyl]-acetamide
3-Methoxy-N-[4-methyl-3-(5-pyridin-3-yl-oxazol-2-ylamino)-phenyl]-propionamide
1-(4-Cyano-phenyl)-3-[4-methyl-3-(5-pyridin-3-yl-oxazol-2-ylamino)-phenyl]-urea
1-(4-Fluoro-phenyl)-3-[4-methyl-3-(5-pyridin-3-yl-oxazol-2-ylamino)-phenyl]-urea
1-(2-Fluoro-phenyl)-3-[4-methyl-3-(5-pyridin-3-yl-oxazol-2-ylamino)-phenyl]-urea
1-[4-Methyl-3-(5-pyridin-3-yl-oxazol-2-ylamino)-phenyl]-3-(4-trifluoromethyl-phenyl)-urea
1-(4-Chloro-phenyl)-3-[4-methyl-3-(5-pyridin-3-yl-oxazol-2-ylamino)-phenyl]-urea
1-[4-Methyl-3-(5-phenyl-oxazol-2-ylamino)-phenyl]-3-(3-trifluoromethyl-phenyl)-urea
1-(4-Cyano-phenyl)-3-[4-methyl-3-(5-pyridin-3-yl-oxazol-2-ylamino)-phenyl]-thiourea
1-(4-Cyano-phenyl)-3-[4-methyl-3-(5-pyridin-4-yl-oxazol-2-ylamino)-phenyl]-thiourea
(2-{2-Methyl-5-[3-(4-trifluoromethyl-phenyl)-ureido]-phenylamino}-oxazol-5-yl)-acetic acid ethyl ester
1-Benzyl-3-[4-methyl-3-(5-pyridin-4-yl-oxazol-2-ylamino)-phenyl]-thiourea
4-(4-Methyl-piperazin-1-ylmethyl)-N-[4-methyl-3-(5-pyridin-3-yl-oxazol-2-ylamino)-phenyl]-benzamide
3-Dimethylamino-N-[4-methyl-3-(5-pyridin-3-yl-oxazol-2-ylamino)-phenyl]-benzamide
3-Bromo-N-[4-methyl-3-(5-pyridin-3-yl-oxazol-2-ylamino)-phenyl]-benzamide
N-[4-Methoxy-3-(5-pyridin-3-yl-oxazol-2-ylamino)-phenyl]-3-trifluoromethyl-benzamide
4-(3-Dimethylamino-propylamino)-N-[4-methyl-3-(5-pyridin-3-yl-oxazol-2-ylamino)-phenyl]-3-trifluoromethyl-benzamide
N-[4-Fluoro-3-(5-pyridin-3-yl-oxazol-2-ylamino)-phenyl]-3-trifluoromethyl-benzamide
1H-Indole-6-carboxylic acid [4-methyl-3-(5-pyridin-4-yl-oxazol-2-ylamino)-phenyl]-amide
3-Isopropoxy-N-[4-methyl-3-(5-pyridin-4-yl-oxazol-2-ylamino)-phenyl]-benzamide
N-[4-Methyl-3-(5-pyridin-2-yl-oxazol-2-ylamino)-phenyl]-3-trifluoromethyl-benzamide 3,5-Dimethoxy-N-[4-methyl-3-(5-pyridin-4-yl-oxazol-2-ylamino)-phenyl]-benzamide
N-[3-(5-Pyridin-3-yl-oxazol-2-ylamino)-phenyl]-3-trifluoromethyl-benzamide
N-[4-Methyl-3-(5-phenyl-oxazol-2-ylamino)-phenyl]-3-trifluoromethyl-benzamide
3-Fluoro-4-(4-methyl-piperazin-1-ylmethyl)-N-[4-methyl-3-(5-pyridin-3-yl-oxazol-2-ylamino)-phenyl]-benzamide
N-[4-Chloro-3-(5-pyridin-3-yl-oxazol-2-ylamino)-phenyl]-3-trifluoromethyl-benzamide
N-[4-Methyl-3-(5-pyridin-3-yl-oxazol-2-ylamino)-phenyl]-terephthalamide
5-Methyl-isoxazole-4-carboxylic acid [4-methyl-3-(5-pyridin-4-yl-oxazol-2-ylamino)-phenyl]-amide
4-Cyano-N-[4-methyl-3-(5-pyridin-4-yl-oxazol-2-ylamino)-phenyl]-benzamide
N-[4-Methyl-3-(5-pyridin-3-yl-oxazol-2-ylamino)-phenyl]-isonicotinamide
N-[4-Methyl-3-(4-pyridin-3-yl-oxazol-2-ylamino)-phenyl]-3-trifluoromethyl-benzamide
[4-Methyl-3-(5-pyridin-3-yl-oxazol-2-ylamino)-phenyl]-carbamic acid isobutyl ester
(5-Isobutoxycarbonylamino-2-methyl-phenyl)-(5-pyridin-3-yl-oxazol-2-yl)-carbamic acid isobutyl ester
[4-Methyl-3-(5-pyridin-4-yl-oxazol-2-ylamino)-phenyl]-carbamic acid isobutyl ester
N-[4-Methyl-3-(5-pyridin-4-yl-oxazol-2-ylamino)-phenyl]-2-nm-tolyl-acetamide
2-(4-Fluoro-phenyl)-N-[4-methoxy-3-(5-pyridin-4-yl-oxazol-2-ylamino)-phenyl]-acetamide
2-(2,4-Difluoro-phenyl)-N-[4-methyl-3-(5-phenyl-oxazol-2-ylamino)-phenyl]-acetamide
2-(3-Bromo-phenyl)-N-[4-methyl-3-(5-pyridin-2-yl-oxazol-2-ylamino)-phenyl]-acetamide
3-(4-Fluoro-phenyl)-N-[4-methyl-3-(5-pyridin-4-yl-oxazol-2-ylamino)-phenyl]-propionamide
N-{3-[5-(4-Cyano-phenyl)-oxazol-2-ylamino]-4-methyl-phenyl}-2-(2,4-difluoro-phenyl)-acetamide
4-Methyl-pentanoic acid [4-methyl-3-(5-pyridin-3-yl-oxazol-2-ylamino)-phenyl]-amide
N-[4-Methyl-3-(5-pyridin-3-yl-oxazol-2-ylamino)-phenyl]-2-piperazin-1-yl-acetamide
N-[4-Methyl-3-(5-pyridin-3-yl-oxazol-2-ylamino)-phenyl]-3-piperazin-1-yl-propionamide
2-(2,6-Dichloro-phenyl)-N-[4-methyl-3-(5-pyrid-4-yl-oxazol-2-ylamino)-phenyl]-acetamide
N-[4-Methyl-3-(5-pyridin-3-yl-oxazol-2-ylamino)-phenyl]-3-pyrrolidin-1-yl-propionamide
N-[4-Methoxy-3-(5-pyridin-4-yl-oxazol-2-ylamino)-phenyl]-2-(4-trifluoromethyl-phenyl)-acetamide
2-(4-Methoxyphenyl)-N-[4-methyl-3-(5-pyridin-4-yl-oxazol-2-ylamino)-phenyl]-acetamide
N-(4-Cyano-phenyl)-4-methyl-3-(5-pyridin-3-yl-oxazol-2-ylamino)-benzamide
N-(3-Dimethylamino-phenyl)-4-methyl-3-(5-pyridin-4-yl-oxazol-2-ylamino)-benzamide
N-(2-Dimethylamino-ethyl)-4-methyl-3-(5-pyridin-3-yl-oxazol-2-ylamino)-benzamide
N-(3-Fluoro-4-methyl-phenyl)-4-methyl-3-(5-pyridin-4-yl-oxazol-2-ylamino)-benzamide
N-(3-Chloro-phenyl)-4-methyl-3-(5-pyridin-3-yl-oxazol-2-ylamino)-benzamide
N-Benzyl-4-methyl-3-(5-pyridin-4-yl-oxazol-2-ylamino)-benzamide
N-(4-Methoxy-benzyl)-4-methyl-3-(5-pyridin-4-yl-oxazol-2-ylamino)-benzamide
[4-Methyl-3-(5-pyridin-4-yl-oxazol-2-ylamino)-phenyl]-morpholin-4-yl-methanone
[4-Methyl-3-(5-pyridin-4-yl-oxazol-2-ylamino)-phenyl]-piperazin-1-yl-methanone
N-(4-Fluoro-phenyl)-2-[4-methyl-3-(5-pyridin-4-yl-oxazol-2-ylamino)-phenyl]-acetamide

### Process for Manufacturing a Compound of Formula III Depicted Above.

This entails the condensation of a substrate of general formula 10 with a thiourea of the type 11.

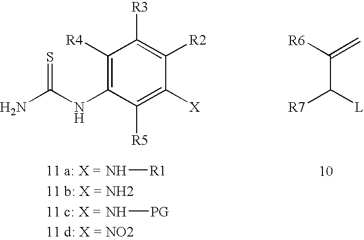

Substituent “V” in formula 10 is a nucleofugal leaving group in nucleophilic substitution reactions (for example, L can be selected from chloro, bromo, iodo, toluenesulfonyloxy, methanesulfonyloxy, trifluoromethanesulfonyloxy, etc., with L being preferentially a bromo group).

Group R1 in formula 11a corresponds to group R1 as described in formula III.

Group “PG” in formula 11c is a suitable protecting group of a type commonly utilized by the person skilled in the art.

The reaction of 10 with 1 a-d leads to a thiozole-type product of formula 12a-d.

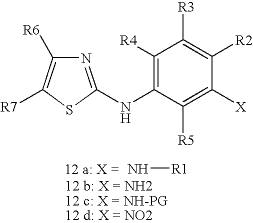

Formula 12a is the same as formula I. Therefore, R1 in 12a corresponds to R1 in formula III.

Formula 12b describes a precursor to compounds of formula III which lack substituent R1. Therefore, in a second phase of the synthesis, substituent R1 is connected to the free amine group in 12b, leading to the complete structure embodied by formula III:

12b+“R1”→III

The introduction of R1, the nature of which is as described on page 3 for the general formula III, is achieved by the use of standard reactions that are well known to the person skilled in the art, such as alkylation, acylation, sulfonylation, formation of ureas, etc.

Formula 12c describes an N-protected variant of compound 12b. Group “PG” in formula 12c represents a protecting group of the type commonly utilized by the person skilled in the art. Therefore, in a second phase of the synthesis, group PG is cleaved to transform compound 12c into compound 12b. Compound 12b is subsequently advanced to structures of formula I as detailed above.

Formula 12d describes a nitro analogue of compound 12b. In a second phase of the synthesis, the nitro group of compound 12d is reduced by any of the several methods utilized by the person skilled in the art to produce the corresponding amino group, namely compound 12b. Compound 12b thus obtained is subsequently advanced to structures of formula III as detailed above.

Examples of compound synthesis is found in our previous applications WO 2004/014903 and U.S. 60/513,214, incorporated herein by reference.

The method according to the invention includes preventing, delaying the onset and/or treating inflammatory muscle disorders including myositis and muscular dystrophy and associated damages in humans.

In the method defined above, any compound capable of depleting mast cells can be used. Such compounds can belong to, as explicated above, tyrosine kinase inhibitors, such as c-kit inhibitors, but are not limited to any particular family so long as said compound shows capabilities to deplete mast cells. Depletion of mast cells can be evaluated using for example one of the mast cell lines depicted above using routine procedure. Best compounds are compounds exhibiting the greatest selectivity.

In a further embodiment, c-kit inhibitors as mentioned above are inhibitors of wild type or mutant activated c-kit. In this regard, the invention contemplates a method for treating inflammatory muscle disorders including myositis and muscular dystrophy comprising administering to a human in need of such treatment a compound that is a selective, potent and non toxic inhibitor of c-kit obtainable by a screening method which comprises:

a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild. Alternatively, in step a) activated c-kit is SCF-activated c-kit wild.

A best mode for practicing this method consists of testing putative inhibitors at a concentration above 10 μM in step a). In step c), IL-3 is preferably present in the culture media of IL-3 dependent cells at a concentration comprised between 0.5 and 10 ng/ml, preferably between 1 to 5 ng/ml. These screening may be performed following our previous application WO 03/003006, which is incorporated herein by reference.

Therefore, the invention embraces the use of the compounds defined above to manufacture a medicament for treating inflammatory muscle disorders such as myositis including polymyositis (PM), dermatomyositis (DM) and inclusion body myositis (IBM) as well as all forms of muscular dystrophy including Duchenne (DMD), Becker, Facioscapulohumeral, Limb-Girdle, Myotonic, Congenital, Distal, Emery-Dreifuss and Oculopharyngeal Muscular Dystrophies.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

More particularly, the invention relates to a pharmaceutical composition intended for oral administration.

Pharmaceutical compositions suitable for use in the invention include compositions wherein compounds for depleting mast cells, such as c-kit inhibitors, or compounds inhibiting mast cells degranulation are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred.

### Example 1

### AB Compounds of Formula III, IV, V and X are Selective and Potent c-Kit and Mast Cell Inhibitors

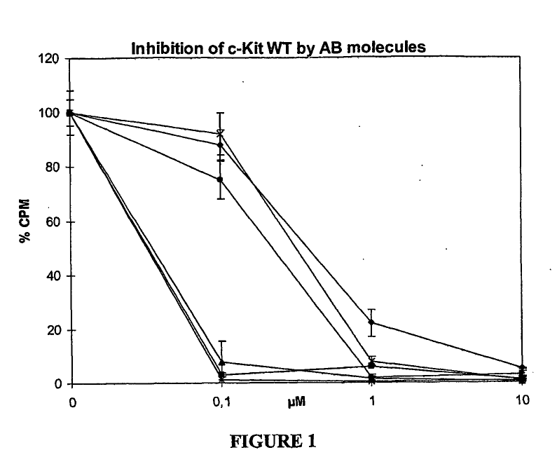

The specific compounds as listed above are non limitative illustrative examples of AB compounds. They display IC50 below 5 μM, 1 μM or even 0.1 μM on different forms of c-KIT (FIG. 1). Also, these AB compounds are selective for c-KIT versus other tyrosine kinases (Table 1).

TABLE 1
Inhibition of various protein tyrosine kinases by the
AB compound in vitro

| Enzyme/Cell line | |
|---|---|
| In vitro enzymatic assay | |
| on purified kinases | IC50 [μM] |
| c-Kit | 0.01 |
| PDGF-beta | 0.49 |
| ABL1 | 5.7 |
| VEGFR1 | IC50 > 100 |
| EGFR | IC50 > 100 |
| FGFR1 | IC50 > 100 |
| FLT3 | IC50 > 100 |
| JAK2 | IC50 > 100 |
| AKT1 | 57 |
| PKC-alpha | 100 |
| SRC | IC50 > 100 |
| IGF1R | IC50 > 100 |
| PIM1 | 19 |

In addition, the AB compounds potently and dose-dependently inhibited the growth of the mast cells (MC) when they were cultured in the presence of SCF (with an IC50 of <0.1 μM). Again these in vitro data confirmed the potent and selective inhibitory activity of c-Kit tyrosine kinase activity as well as the ability of the AB compound to inhibit almost completely the survival of MC population at concentration lower than 0.1 μM. AB compounds have also been shown to deplete mast cells in vivo. The AB compound has successfully completed preclinical development in September 2003. Safety pharmacology studies revealed no significant effects of the AB compound on the central nervous, cardiovascular and respiratory systems.

1. A method for treating an inflammatory muscle disorder, comprising administering to a human in need thereof a compound capable of depleting mast cells or a compound inhibiting mast cells degranulation.

2. The method according to claim 1 comprising administering a c-kit inhibitor to a human in need of such treatment.

3. The method according to claim 2, wherein said c-kit inhibitor is a non-toxic, selective and potent c-kit inhibitor unable to promote death of IL-3 dependent cells cultured in presence of IL-3.

4. The method according to claim 3, wherein said c-kit inhibitor is selected from the group consisting of:
2-(3-Substitutedaryl)amino-4-aryl-thiazoles,
2-aminoaryloxazoles,
pyrimidine derivatives,
indolinone derivatives,
monocyclic, bicyclic aryl and heteroaryl compounds,
and quinazoline derivatives.

5. The method according to claim 4, wherein said c-kit inhibitor is selected from compounds belonging to the 2-(3-Substitutedaryl)amino-4-aryl-thiazoles having formula III: 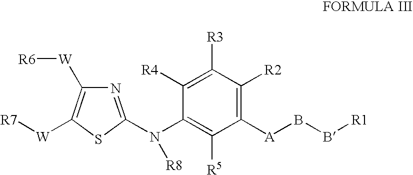
wherein
R⁶ and R⁷ are independently from each other chosen from one of the following:
i) H, F, Cl, Br and I;
ii) an alkyl¹ group defined as a linear, branched or cycloalkyl group containing from 1 to 10 carbon atoms, and optionally substituted with one or more heteroatoms selected from F, Cl, Br I, oxygen, and nitrogen, wherein the nitrogen heteroatom is optionally in the form of a pendant basic nitrogen functionality; trifluoromethyl, carboxyl, cyano, nitro, and formyl;
(iii) an aryl¹ group defined as phenyl or a substituted variant thereof that contains one or more substituents selected from
I, F, Cl and Br;
an alkyl¹ group;
a cycloalkyl, aryl or heteroaryl group optionally substituted with a pendant basic nitrogen functionality;
trifluoromethyl, O-alkyl¹, carboxyl, cyano, nitro, formyl, hydroxy, NH-alkyl¹, N(alkyl¹)(alkyl¹), and amino, wherein each of the NH-alkyl¹, N(alkyl¹)(alkyl¹) and amino substituents is optionally in the form of a basic nitrogen functionality;
(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, which optionally contains one or more substituents selected from
F, Cl, Br and I;
an alkyl¹ group;
a cycloalkyl, aryl or heteroaryl group optionally substituted with a pendant basic nitrogen functionality,
trifluoromethyl, O-alkyl¹, carboxyl, cyano, nitro, formyl, hydroxy, NH-alkyl¹, N(alkyl¹)(alkyl¹), and amino, the latter nitrogen wherein each of the NH-alkyl¹, N(alkyl¹)(alkyl¹) and amino substituents is optionally in the form of a basic nitrogen functionality;
(v) trifluoromethyl, carboxyl, cyano, nitro, formyl, hydroxy, N(alkyl¹)(alkyl¹), and amino, wherein each of the N(alkyl¹)(alkyl¹) and amino substituents is optionally in the form of a basic nitrogen functionality.
R⁸ is selected from
(i) hydrogen,
(ii) a linear or branched alkyl group containing from 1 to 10 carbon atoms and optionally substituted with one or more hetereoatoms selected from F, Cl, Br I, oxygen, and nitrogen, wherein the nitrogen heteroatom is optionally in the form of a pendant basic nitrogen functionality,
(iii) CO—R8, COORS, CONHR8 or SO2R8, wherein R8 is
a linear or branched alkyl group containing from 1 to 10 carbon atoms and optionally substituted with one or more heteroatoms selected from F, Cl, Br, I, oxygen, and nitrogen, wherein the nitrogen heteroatom is optionally in the form of a pendant basic nitrogen functionality,
an aryl group defined as phenyl or a substituted variant thereof that contains one or more substituents selected from F, Cl, Br I, alkyl groups containing from 1 to 10 carbon atoms and optionally substituted with one or more heteroatoms selected from F, Cl, Br I, oxygen, and nitrogen, wherein the nitrogen heteroatom is optionally in the form of a pendant basic nitrogen functionality; trifluoromethyl, C₁₋₆alkyloxy, carboxyl, cyano, nitro, formyl, hydroxy, C₁₋₆alkylamino, di(C₁₋₆alkyl)amino, and amino, wherein each of the C₁₋₆alkylamino di(C₁₋₆alkyl)amino, and amino substituents is optionally in the form of a pendant basic nitrogen functionality; CO—R, COO—R, CONH—R, SO2-R, and SO2NH—R, wherein R is a linear or branched alkyl group containing from 1 to 10 carbon atoms and optionally substituted with at least one heteroatom, selected from F, Cl, Br, I oxygen, and nitrogen, wherein the nitrogen heteroatom is optionally in the form of a pendant basic nitrogen functionality, or
a heteroaryl group defined as a pyridyl, pyrimidinyl, pyrazinyl, pyridazinyl, thienyl, triazolyl, imidazolyl, pyrazolyl, pyrrolyl, furanyl, oxazolyl, isoxazolyl, triazolyl, tetrazolyl, indolyl, benzimidazole, quinolinyl group, the heteroaryl group contains one or more substituents selected from F, Cl, Br, I alkyl groups containing from 1 to 10 carbon atoms and optionally substituted with one or more heteroatoms selected from F, Cl, Br, I oxygen, and nitrogen, wherein the nitrogen heteroatom is optionally in the form of a pendant basic nitrogen functionality; trifluoromethyl, C₁₋₆alkyloxy, carboxyl, cyano, nitro, formyl, hydroxy, C₁₋₆alkylamino, di(C₁₋₆alkyl)amino, and amino, wherein each of the C₁₋₆alkylamino di(C₁₋₆alkyl)amino and amino substituents is optionally in the form of a basic nitrogen functionality; CO—R, COO—R, CONH—R, SO2-R, and SO2NH—R, wherein R is a linear or branched alkyl group containing from 1 to 10 carbon atoms and optionally substituted with at least one heteroatom selected from F, Cl, Br, I oxygen, and nitrogen, wherein the nitrogen heteroatom is optionally in the form of a pendant basic nitrogen functionality;
R2, R3, R4 and R5 each independently are selected from hydrogen, F, Cl, Br, I a linear or branched alkyl group containing from 1 to 10 carbon atoms and optionally substituted with one or more heteroatoms selected from F, Cl, Br, I oxygen, and nitrogen, wherein the nitrogen heteroatom is optionally in the form of a pendant basic nitrogen functionality; trifluoromethyl, C₁₋₆alkyloxy, amino, C₁₋₆alkylamino, di(C₁₋₆alkyl)amino, carboxyl, cyano, nitro, formyl, hydroxy, CO—R, COO—R, CONH—R, SO2-R, and SO2NH—R, wherein R is a linear or branched alkyl group containing from 1 to 10 carbon atoms and optionally substituted with at least one heteroatom selected from F, Cl, Br, I oxygen, and nitrogen, wherein the nitrogen heteroatom is optionally in the form of a pendant basic nitrogen functionality;
A is CH2, O, S, SO2, CO, or COO,
B is a bond or NH, NCH3, NR*, (CH2)n, with n equals 0, 1 or 2, O, S, SO2, CO, or COO,
B′ is a bond or NH, NCH3, NR*, (CH2)n, with n equals 0, 1 or 2, O, S, SO2, CO or COO;
R* being an alkyls, aryl¹ or heteroaryl¹
W is a bond or a linker selected from NH, NHCO, NHCOO, NHCONH, NHSO2, NHSO2NH, CO, CONH, COO, COCH2, (CH2)n, with n equals 0, 1 or 2, CH2-CO, CH2COO, CH2-NH, O, OCH2, S, SO2, and SO2NH;
R¹ is:
a) a linear or branched alkyl group containing from 1 to 10 carbon atoms optionally substituted with at least one heteroatom selected from I, Cl, Br and F, and/or bearing a pendant basic nitrogen functionality;
b) an aryl or heteroaryl group optionally substituted by with an alkyl or aryl group optionally substituted with a heteroatom selected from I, Cl, Br and F or bearing a pendant basic nitrogen functionality;
c) an alkyl¹, aryl¹ or heteroaryl¹.

6. A method according to claim 5, wherein said c-kit inhibitor is selected from compounds having formula V: 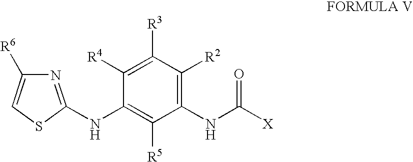
wherein X is R or NRR′ and wherein R and R′ are independently chosen from
H,
an aryl, a heteroaryl, an alkyl, or a cycloalkyl group optionally substituted with at least one heteroatom selected from F, I, Cl and Br and optionally bearing a pendant basic nitrogen functionality;
an aryl, a heteroaryl, an alkyl or a cycloalkyl group substituted with an aryl,
a heteroaryl, an alkyl or a cycloalkyl group optionally substituted with at least one heteroatom selected from F, I, Cl and Br and optionally bearing a pendant basic nitrogen functionality,
R² is hydrogen, halogen; a linear or branched alkyl group containing from 1 to 10 carbon atoms; trifluoromethyl or alkoxy;
R³ is hydrogen, halogen; a linear or branched alkyl group containing from 1 to 10 carbon atoms; trifluoromethyl or alkoxy;
R⁴ is hydrogen, halogen; a linear or branched alkyl group containing from 1 to 10 carbon atoms; trifluoromethyl or alkoxy;
R⁵ is hydrogen, halogen; a linear or branched alkyl group containing from 1 to 10 carbon atoms; trifluoromethyl or alkoxy;
R⁶ is one of the following:
(i) an aryl group defined as phenyl or a substituted variant thereof containing one or more substituents selected from halogen, alkyl groups containing from 1 to 10 carbon atoms, trifluoromethyl, and alkoxy;
(ii) a 2, 3, or 4-pyridyl group, which optionally contains one or more substituents selected from halogen, alkyl groups containing from 1 to 10 carbon atoms, trifluoromethyl and alkoxy;
(iii) a five-membered ring aromatic heterocyclic group selected from 2-thienyl, 3-thienyl, 2-thiazolyl, 4-thiazolyl, 5-thiazolyl, wherein the five-membered ring aromatic group optionally contains one or more substituents selected from halogen, an alkyl group containing from 1 to 10 carbon atoms, trifluoromethyl, and alkoxy;
(iv) H, I, F, Cl, Br; NH2, NO2 or SO2-R, wherein R is a linear or branched alkyl group containing 1 to 10 carbon atoms, and optionally substituted with at least one heteroatom selected from I, Cl, Br and F, and/or bearing a pendant basic nitrogen functionality.

7. The method according to claim 4, wherein said c-kit inhibitor is selected from 2-aminoaryloxazoles of formula X: 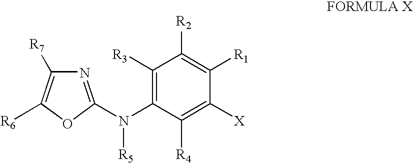
wherein substituents R1-R7 and X are defined as follows:
R1, R2, R3 and R4 each independently are selected from hydrogen, F, Cl, Br, I a linear or branched alkyl group containing from 1 to 10 carbon atoms and optionally substituted with one or more heteroatoms selected from F, Cl, Br, I oxygen, and nitrogen, wherein the nitrogen heteroatom is optionally in the form of a pendant basic nitrogen functionality; trifluoromethyl, C₁₋₆alkyloxy, amino, C₁₋₆alkylamino, di(C₁₋₆alkyl)amino, carboxyl, cyano, nitro, formyl, hydroxy, CO—R, COO—R, CONH—R, SO2-R, and SO2NH—R, wherein R is a linear or branched alkyl group containing from 1 to 10 carbon atoms and optionally substituted with at least one heteroatom selected from F, CI, Br, I oxygen, and nitrogen, wherein the nitrogen heteroatom is optionally in the form of a pendant basic nitrogen functionality;
R5 is one of the following:
(i) hydrogen,
(ii) a linear or branched alkyl group containing from 1 to 10 carbon atoms and optionally substituted with one or more heteroatoms selected from F, Cl, Br, I, oxygen, and nitrogen, wherein the nitrogen heteroatom is optionally in the form of a pendant basic nitrogen functionality,
(iii) CO—R8, COOR8, CONHR8 or SO2R8, wherein R8 is
a linear or branched alkyl group containing from 1 to 10 carbon atoms and optionally substituted with one or more selected from F, CI, Br, I oxygen, and nitrogen, wherein the nitrogen heteroatom is optionally in the form of a pendant basic nitrogen functionality,
an aryl group defined as phenyl or a substituted variant thereof containing one or more substituents selected from F, Cl, Br, I alkyl groups containing from 1 to 10 carbon atoms and optionally substituted with one or more heteroatoms selected from F, Cl, Br I, oxygen, and nitrogen, wherein the nitrogen heteroatom is optionally in the form of a pendant basic nitrogen functionality; as trifluoromethyl, C₁₋₆alkyloxy, carboxyl, cyano, nitro, formyl, hydroxy, C₁₋₆alkylamino, di(C₁₋₆alkyl)amino, and amino, wherein each of the C₁₋₆alkylamino, di(C₁₋₆alkyl)amino and amino substituents is optionally in the form of a pendant basic nitrogen functionality; CO—R, COO—R, CONH—R, SO2-R, and SO2NH—R, wherein R is a linear or branched alkyl group containing from 1 to 10 carbon atoms and optionally substituted with at least one heteroatom selected from F, Cl, Br, I oxygen, and nitrogen, wherein the nitrogen heteroatom is optionally in the form of a pendant basic nitrogen functionality, or
a heteroaryl group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, optionally containing one or more substituents selected from F, Cl, Br, I alkyl groups containing from 1 to 10 carbon atoms and optionally substituted with one or more heteroatoms selected from F, Cl, Br, I oxygen, and nitrogen, wherein the nitrogen heteroatom is optionally in the form of a pendant basic nitrogen functionality; trifluoromethyl, C₁₋₆alkyloxy, carboxyl, cyano, nitro, formyl, hydroxy, C₁₋₆alkylamino, di(C₁₋₆alkyl)amino, and amino, wherein each of the C₁₋₆alkylamino, di(C₁₋₆alkyl)amino and amino substituents is optionally in the form of a basic nitrogen functionality; CO—R, COO—R, CONH—R, SO2-R, and SO2NH—R, wherein R is a linear or branched alkyl group containing from 1 to 10 carbon atoms and optionally substituted with at least one heteroatom selected from F, Cl, Br, I oxygen, and nitrogen, wherein the nitrogen heteroatom is optionally in the form of a pendant basic nitrogen functionality;
R6 and R7 each independently are selected from:
(i) H, F, Cl Br and I;
(ii) an alkyl¹ group defined as a linear, branched or cycloalkyl group containing from 1 to 10 carbon atoms and optionally substituted with one or more heteroatoms selected from F, Cl, Br, I oxygen, and nitrogen, wherein the nitrogen heteroatom is optionally in the form of a pendant basic nitrogen functionality; trifluoromethyl, carboxyl, cyano, nitro, formyl; CO—R, COO—R, CONH—R, SO2-R, and SO2NH—R, wherein R is a linear or branched alkyl group containing 1 to 10 carbon atoms and optionally substituted with at least one heteroatom selected from F, Cl, Br, I oxygen, and nitrogen, wherein the nitrogen heteroatom is optionally in the form of a pendant basic nitrogen functionality; cycloalkyl or aryl or heteroaryl group optionally substituted with a pendant basic nitrogen functionality,
(iii) an aryl¹ group defined as phenyl or a substituted variant thereof containing one or more substituents selected from
I, F, Cl, and Br;
an alkyl¹ group;
a cycloalkyl, aryl or heteroaryl group optionally substituted with a pendant basic nitrogen functionality;
trifluoromethyl, O-alkyl¹, carboxyl, cyano, nitro, formyl, hydroxy, NH-alkyl, N(alkyl¹)(alkyl¹), and amino, wherein each of the NH-alkyl, N(alkyl¹)(alkyl¹) and amino substituents is optionally in the form of a basic nitrogen functionality;
NHCO—R, NHCOO—R, NHCONH—R, NHSO2-R, NHSO2NH—R, CO—R, COO—R, CONH—R, SO2-R or SO2NH—R, wherein R corresponds to hydrogen, alkyl¹, aryl or heteroaryl,
(iv) a heteroaryl¹ group defined as a pyridyl, pyrimidinyl, pyrazinyl, pyridazinyl, thienyl, thiazolyl, imidazolyl, pyrazolyl, pyrrolyl, furanyl, oxazolyl, isoxazolyl, triazolyl, tetrazolyl, indolyl, benzimidazole, quinolinyl group, said heteroaryl¹ group optionally contains one or more substituents selected from
F, Cl, Br and I;
an alkyl¹ group;
a cycloalkyl, aryl or heteroaryl group optionally substituted with a pendant basic nitrogen functionality,
trifluoromethyl, O-alkyl¹, carboxyl, cyano, nitro, formyl, hydroxy, NH-alkyl¹, N(alkyl¹)(alkyl¹), and amino, wherein each of the NH-alkyl¹, N(alkyl¹)(alkyl¹) and amino substituents is optionally in the form of a basic nitrogen functionality;
NHCO—R, NHCOO—R, NHCONH—R, NHSO2-R, NHSO2NH—R, CO—R, COO—R, CONH—R, SO2-R or SO2NH—R, wherein R corresponds to hydrogen or alkyl¹,
(v) an O-aryl¹, NH-aryl¹, O-heteroaryl¹ or NH-heteroaryl¹ group;
(vi) trifluoromethyl, O-alkyl¹, carboxyl, cyano, nitro, formyl, hydroxy, NH-alkyl¹, N(alkyl¹)(alkyl¹), and amino, wherein each of the NH-alkyl¹, N(alkyl¹)(alkyl¹) and amino substituents is optionally in the form of a basic nitrogen functionality, or
(vi) NHCO—R, NHCOO—R, NHCONH—R, NHSO2-R, NHSO2NH—R, CO—R, COO—R, CONH—R, SO2-R or SO2NH—R, wherein R corresponds to hydrogen, aryl or heteroaryl;
X is
NR9R10, wherein R9 and/or R10 are each hydrogen or
i) alkyl¹ group, CF3
ii) an aryl¹, heteroaryl¹ or cycloalkyl group optionally substituted with a pendant basic nitrogen functionality,
iii) a CO—R, COO—R, CON—RR′ or SO2R, where R and R′ are a hydrogen, alkyl¹, aryl¹ or heteroaryl¹, optionally substituted with a pendant basic nitrogen functionality;
or
CO—NR9R10, wherein R9 and/or R10 are hydrogen or
i) an alkyl¹ group, CF3 or
ii) an aryl¹, heteroaryl¹ or cycloalkyl group optionally substituted with a pendant basic nitrogen functionality.

8. The method according to claim 4, wherein said c-kit inhibitor is selected from the group consisting of N-phenyl-2-pyrinaidine-amine derivatives having the formula II: 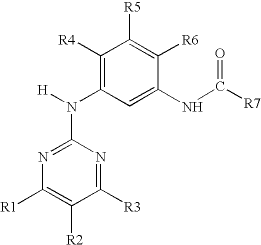
wherein R1, R2 and R3 are independently selected from H, F, Cl, Br, I, a C₁-C₅ alkyl and a cyclic or heterocyclic group,
R4, R5 and R6 are independently selected from H, F, Cl, Br, I, and a C1-C5 alkyl,
and R7 is a phenyl group containing at least one substituent, which possesses at least one basic site.

9. The method according to claim 8, wherein said c-kit inhibitor is the 4-(4-méthylpipérazine-1-ylméthyl)-N-[4-méthyl-3-(4-pyridine-3-yl)pyrimidine-2ylamino)phényl]-benzamide.

10. A method for treating an inflammatory muscle disorder comprising administering to a human in need of such treatment a compound that is a selective, potent and non toxic inhibitor of activated c-kit, said compound is produced by a screening method which comprises:
a) bringing into contact (i) activated c-kit and (ii) one or more compounds to be tested under conditions allowing the activated c-kit and the one or more compound to be tested to form a complex,
b) selecting from the one or more compounds a subset of compounds that inhibit activated c-kit, and
c) selecting from the subset of compounds a compound, that is unable to promote death of IL-3 dependent cells cultured in presence of IL-3, as the selective, potent and non toxic inhibitor of activated c-kit.

11-12. (canceled)

13. The method of claim 1, wherein the inflammatory muscle disorder is myositis or muscular dystrophy.

14. The method of claim 13, wherein the myositis is selected from the group consisting of polymyositis (PM), dermamyositis (DM) and inclusion body myositis (IBM) and the muscular dystrophy is selected from the group consisting of Duchenne (DMD), Becker, Facioscapulohumeral, Limb-Girdle, Myotonic, Congenital, Distal, Emery-Dreifuss and Oculopharyngeal Muscular Dystrophies.

15. The method of claim 10, wherein the inflammatory muscle disorder is myositis or muscular dystrophy.

16. The method of claim 15, wherein the myositis is selected from the group consisting of polymyositis (PM), dermamyositis (DM) and inclusion body myositis (IBM) and the muscular dystrophy is selected from the group consisting of Duchenne (DMD), Becker, Facioscapulohumeral, Limb-Girdle, Myotonic, Congenital, Distal, Emery-Dreifuss and Oculopharyngeal Muscular Dystrophies.
